(19) United States
(12) Patent Application Publication
(10) Pub. No.: US 2024/0191187 A1
(43) Pub. Date: Jun. 13, 2024

(54) MEDIA AND METHODS FOR GROWING MAMMARY ORGANOIDS

(71) Applicant: STEMCELL TECHNOLOGIES CANADA INC., Vancouver (CA)

(72) Inventors: David ROWBOTHAM, Vancouver (CA); John STINGL, Vancouver (CA)

(21) Appl. No.: 18/555,332

(22) PCT Filed: Apr. 14, 2022
(86) PCT No.: PCT/CA2022/050583

§ 371 Date: Oct. 13, 2023

Related U.S. Application Data
(60) Provisional application No. 63/175,686, filed on Apr. 16, 2021.

(51) Int. Cl. C12N 5/071

(52) U.S. Cl. CPC C12N 5/0631; C12N 2501/11; C12N 2501/15; C12N 2501/155; C12N 2501/195; C12N 2501/415; C12N 2501/727; C12N 2513/00

(57) ABSTRACT

Disclosed are media, kits and methods for directed formation of mammary organoids. Embodiments of organoid media may be used to bias/enrich the formation of luminal organoids from isolated mammary epithelial cells. Embodiments of modified organoid media may be used to bias/enrich the formation of mixed lineage organoids from isolated mammary epithelial cells, or to subvert luminal organoids to mixed lineage organoids.

### CROSS-REFERENCE TO RELATED APPLICATION

This application claims the benefit of priority to U.S. Provisional Application No. 63/175,686 filed Apr. 16, 2021, the contents of which are incorporated herein by reference in their entirety.

### FIELD

This disclosure relates to cell culture applications, and more specifically to cell culture applications using cells of the mammary gland, and still more specifically to cell culture applications related to growing multicellular structures composed of specific mammary gland cell types.

### BACKGROUND

Approximately 1 in 8 US women will be diagnosed with invasive breast cancer over the course of her lifetime. Breast tumors originate in the epithelium of the mammary gland. The mammary epithelium is composed of a series of branched ducts that, during lactation, drain milk-producing alveoli. The cells of these ducts and alveoli are organized as a bilayered epithelium with an inner luminal layer and an outer basal layer. The luminal layer is made up of two lineages of epithelial cells: the estrogen receptor (ER) expressing lineage and the milk lineage. The milk lineage cells in resting mammary gland are the cells that will proliferate during pregnancy and generate sac-like structures called alveoli. The cells lining the alveoli, the alveolar cells, will synthesize and secrete milk during lactation. The basal cell layer of the mammary epithelium is composed of basal cells, and since these cells have contractile properties (they squeeze milk out of the mammary gland), they are also called myoepithelial cells.

Each of the three lineages of cells (basal, ER⁺ and milk) in the postnatal gland are largely maintained by their own stem cell population. The ER⁺ lineage express high levels of the luminal keratins (K) 8 and 18, but do not express the basal K5 and K14. These cells, as their name implies, express high levels of ER, as well as the progesterone receptor (PR). The basal cells do not express K8 or K18, but do express keratins K5 and K14. The milk lineage has an intermediate phenotype; in the human, these milk lineage cells express both luminal and basal keratins. The milk lineage in the mouse has a predominantly luminal phenotype, albeit with luminal keratin expression at lower levels, but will have low but detectable transcript levels of basal keratins. The milk lineage does not express ER.

Human breast tumors can be broadly categorized into 3 subtypes based on phenotype: ER⁺, HER2 amplified (HER2⁺), and triple negative (ER⁻, PR⁻, HER2⁻). The triple negative category can be further subdivided into basal-like and claudin^{low}. Although the cellular origins of human breast tumors is still not fully understood, one hypothesis is that ER⁺ tumors originate in ER⁺ cells, HER2⁺ tumors originate in ER⁺ and milk lineage cells, basal-like tumors originate in milk lineage cells, and claudin^{low} tumors originate in basal cells.

In vitro model systems would be beneficial to studying questions of basic mammary gland biology and also to understanding the differences between normal breast cells compared to breast tumor cells. Further, systems could benefit compound screening assays and toxicity studies.

There remains a need for effective in vitro model systems for studying normal and disease-states of mammary glands. In particular, there remains a need for in vitro model systems to study individual cell types of the mammary gland in isolation, whether in the normal or diseased context. Further, there remains a need for ascertained culture conditions, including cell culture media, to obtain in vitro model systems that recapitulate the particular structure or cell type being investigated, whether in a normal or diseased state.

### SUMMARY

This disclosure relates to media, kits and methods for growing mammary organoids. More specifically, this disclosure relates to culturing mammary epithelial cells to predictably and reproducibly yield mammary organoids composed of desired mammary gland cell types.

In one broad aspect of this disclosure are provided methods of forming mammary organoids from isolated mammary epithelial cells. Such methods may comprise contacting the mammary epithelial cells with an organoid medium free of one or both of an exogenously-added WNT signaling agonist and/or an inhibitor of BMP signaling, and culturing the mammary epithelial cells in the organoid medium for a time sufficient to form a first population of organoids enriched for organoids composed of more luminal cells than non-luminal cells (e.g basal cells, stromal cells, etc) then if one or both of the exogenously-added WNT signaling agonist and/or the BMP signaling inhibitor are included in the organoid medium. In one embodiment, the mammary epithelial cells comprise mammary epithelial stem or progenitor cells. In one embodiment, the mammary epithelial cells are isolated from a primate or a rodent. In one embodiment, non-luminal cells are one or more of basal cells, stromal cells, hematopoietic cells, and endothelial cells.

In one embodiment, the luminal cells express K8. In one embodiment, luminal cells co-express K8 and K18. In one embodiment, basal cells express K5. In one embodiment, basal cells co-express K5 and K14.

In one embodiment, the organoid medium comprises a basal medium and one or both of a ligand of ERBB1 and/or a ligand of ERBB4. In one embodiment, the ligand of ERBB1 is not EGF or TGFalpha. In one embodiment, the ligand of ERBB1 is amphiregulin. In one embodiment, the ligand of ERBB4 is also a ligand of a different ERBB receptor family member. In one embodiment, the ligand of ERBB4 is heregulin and/or neuregulin 3.

In one embodiment, the organoid medium includes the inhibitor of BMP signaling but not the WNT signaling agonist. In one embodiment, the organoid medium includes the WNT signaling agonist but not the inhibitor of BMP signaling.

In one embodiment, the WNT signaling agonist (not included in the organoid medium) is an R-spondin, a WNT protein, or an engineered mimetic of either of the foregoing.

In one embodiment, the inhibitor of BMP signaling is a protein or a small molecule. In one embodiment, the inhibitor of BMP signaling is one or more of Noggin, chordin, follistatin, LDN193189, or dorsomorphin.

In one embodiment, the organoid medium is free of an exogenously added sex hormone. In one embodiment, the sex hormone is progesterone.

In one embodiment, the organoids are composed of 50% or more luminal cells.

In one embodiment, the methods may further comprise culturing the first population of organoids in a modified organoid medium to subvert the first population of organoids into a second population of organoids.

In one embodiment, the modified organoid medium is supplemented with EGF. In one embodiment, the modified organoid medium is supplemented with a WNT signaling agonist and/or an inhibitor of BMP signaling.

In one embodiment, the second population of organoids are composed of more basal cells fewer luminal cells then if one or both of the WNT signaling agonist and/or the inhibitor of BMP signaling are not added to the organoid medium.

In one embodiment, the first population of organoids are passageable 5 or more times in the organoid medium. In one embodiment, the second population of organoids are passageable 5 or more times in the modified organoid medium.

In one embodiment, the methods may further comprise contacting the first population or the second population of organoids with an inhibitor of TGF-beta. In one embodiment, the methods may further comprise obtaining nuclear localization of ER on treatment with an inhibitor of TGF-beta.

In one embodiment, the first population of organoids are composed on average of greater than 50% luminal cells and less than 30% basal cells.

In another broad aspect of this disclosure are provided mammary organoid medium formulations. Such mammary organoid media comprise a basal medium and one or both of a ligand of ERBB1 and a ligand of ERBB4; and lacking one or both of an exogenously-added WNT signaling agonist and an inhibitor of BMP signaling.

In one embodiment, the organoid medium includes the inhibitor of BMP signaling but not the WNT signaling agonist. In one embodiment, the organoid medium includes the WNT signaling agonist but not inhibitor of BMP signaling.

In one embodiment, the WNT signaling agonist (not included in the organoid medium) is one or more of an R-spondin, a WNT protein, or an engineered mimetic of either of the foregoing.

In one embodiment, the inhibitor of BMP signaling (not included in the organoid medium) is a protein or a small molecule. In one embodiment, the inhibitor of BMP signaling (not included in the organoid medium) is one or more of Noggin, chordin, follistatin, LDN193189, or dorsomorphin.

In one embodiment, the ligand of ERBB1 is not EGF or TGFalpha. In one embodiment, the ligand of ERBB1 is amphiregulin. In one embodiment, the ligand of ERBB4 is also a ligand of a different ERBB receptor family member. In one embodiment, the ligand of ERBB4 is neuregulin 1 and/or neuregulin 3.

In one embodiment, the organoid medium is free of an exogenously added sex hormone. In one embodiment, the sex hormone is progesterone.

In one embodiment, culturing isolated mammary epithelial cells in the organoid medium enriches organoids composed of more luminal cells than non-luminal cells.

In one embodiment, culturing isolated mammary epithelial cells in the organoid medium enriches organoids composed of more luminal cells and fewer basal cells then if the isolated mammary epithelial cells are cultured in an organoid medium that contains one or both of the exogenously-added WNT signaling agonist and/or the BMP signaling inhibitor. In one embodiment, the mammary epithelial cells comprise mammary epithelial stem or progenitor cells. In one embodiment, the mammary epithelial cells are isolated from a primate or a rodent.

In another broad aspect of this disclosure are provided kits (for formulating media formulations, such as organoid media, modified organoid media, and ER nuclear localization media of this disclosure). In one embodiment, such kits may comprise a basal medium, and a first luminal cell-promoting supplement to be added to the basal medium. In one embodiment, the first supplement may comprise one or both of a ligand of ERBB1 and/or a ligand of ERBB4. In one embodiment, the first supplement may also lack one or both of an exogenously-added WNT signaling agonist and/or an inhibitor of BMP signaling.

In one embodiment, a kit of this disclosure may further comprise a second mixed lineage-promoting supplement to be added to the basal medium, or to the basal medium supplemented with the first supplement. In one embodiment, the second supplement may comprise a second ligand of ERBB1 different from the ligand of ERBB1 in the first supplement. In one embodiment, the second supplement may further comprise one or both of an exogenously-added WNT signaling agonist and/or the inhibitor of BMP signaling.

In one embodiment, a kit of this disclosure may further comprise a third ER nuclear localization supplement to be added to the basal medium, or to the basal medium supplemented with the first supplement and/or the second supplement. In one embodiment, the third supplement may comprise an inhibitor of TGFβ signaling. In one embodiment, the inhibitor of TGFβ signaling is SB431542, RepSox, A77-01, or A83-01.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows representative images of organoids formed from non-sorted mouse mammary epithelial cells. The images in panels A), B) and C) correspond to successive passages of the organoids in an organoid medium that includes an exogenously added WNT agonist and an inhibitor of BMP signaling. The image in panel D shows an image taken by confocal microscopy of passage 2 organoids generated in the organoid medium used to form the organoids depicted in panels A)-C). Organoids were stained for K8, K14, and the nuclear stain DAPI. Scale bars are 500 μm in panels A)-C), and 100 μm in panel D).

FIG. 2 shows representative images of organoids formed from sorted populations of mouse mammary epithelial cells. ER⁺ luminal cells (panels A and D), milk cells (panels B and E), and basal cells (panels C and F) were cultured in an organoid medium free of an inhibitor of BMP signaling, and either in the presence or absence of RSPO-1. Scale bars are 500 μm.

FIG. 3 shows representative images of organoids formed from non-sorted mouse mammary epithelial cells. The images in panel A) show organoids formed in media either including the WNT agonist RSPO-1 or RSPO-3, or lacking an exogenously added WNT signaling agonist. Scale bars are 500 μm. The flow cytometry plots in panel B) show the phenotype among cells formed into organoids in the organoid media of panel A).

FIG. 4 shows representative images of organoids formed from non-sorted mouse mammary epithelial cells across successive passages. Luminal organoids were propagated in an organoid medium of this disclosure either comprising or excluding an exogenously added WNT signaling agonist. Scale bars are 500 μm. Passage 2 organoids were stained for K8, K14, and the nuclear stain DAPI, and imaged by confocal microscopy. Scale bars are 100 μm.

FIG. 5 shows representative images of organoids formed from non-sorted mouse mammary epithelial cells across successive passages. Luminal organoids do not form or propagate in an organoid medium of this disclosure, either comprising or excluding an exogenously added inhibitor of BMP signaling. Scale bars are 500 μm. Passage 2 organoids were stained for K8, K14, and the nuclear stain DAPI, and imaged by confocal microscopy. Scale bars are 100 μm.

FIG. 6 shows representative flow cytometry plots of organoids formed from non-sorted mouse mammary epithelial cells. Panels A) and B) show representative images of organoids after cells were cultured for 14 days (P0) in an R-spondin-containing (panel A) or an R-spondin-free (panel B) medium. Cells of the organoids shown in panels A) and B) were respectively analyzed by flow cytometry for expression of EpCAM and CD49f (panels C) and D)). A summary of flow cytometry data is shown in panel E. Results were confirmed in n=8 mice. Scale bars are 500 μm.

FIG. 7 shows representative images of organoids formed from non-sorted human mammary epithelial cells. Organoids were formed in an organoid medium comprising exogenously added RSPO-1, EGF and NOGGIN. The image in panel A) shows a wider focal area capturing a plurality of formed organoids. Scale bar is 500 μm. The images in panel B) and panel C) respectively show a single organoid imaged under either brightfield, or by confocal microscopy after staining for K8, K14, and the nuclear stain DAPI. Scale bar is 100 μm.

FIG. 8 shows the effects of modulating signaling through a BMP receptor on human organoids formed from non-sorted human mammary epithelial cells. The flow cytometry plots in panel A) compare organoid composition after forming in an organoid medium comprising either a BMP signaling agonist (BMP2 or BMP4) or antagonist (NOGGIN or LDN193189). The flow cytometry plots in panel B) show for two different donors, the effects of inhibiting signaling through a BMP receptor on organoid cell lineage balance.

FIG. 9 shows the effects of modulating signaling through a BMP receptor on human organoids formed from non-sorted human mammary epithelial cells. The plot in panel A) shows the total number of cells included among organoids formed in an organoid medium including either a BMP signaling agonist (BMP2 or BMP4) or antagonist (NOGGIN or LDN193189). The bar graph in panel B) summarizes the composition of organoids formed in organoid media comprising different modulators of BMP signaling. Bars represent mean of 2 experiments +/−standard deviation, as normalized to a control lacking exogenously-added modulators of BMP signaling. In panel C), organoids formed in media either comprising or lacking inhibitors of BMP signaling were imaged by confocal microscopy after staining for K8, K14 and the nuclear stain DAPI. White arrowheads indicate selected regions staining brightly for K8. Scale bar is 50 μm.

FIG. 10 shows images of luminal-biased human organoids taken across successive passages. Organoids were formed in organoid media comprising a combination of two different inhibitors of BMP signaling or in media comprising each BMP signaling inhibitor separately. Scale bar is 500 μm.

FIG. 11 shows the effects of Wnt agonism on organoids formed from non-sorted human mammary epithelial cells. Organoids in panel A) were formed in organoid media either comprising or omitting an agonist of WNT signaling, and representative flow cytometry plots of the composition of such organoids is shown in panel B). Scale bar is 500 μm. The bar graph in panel C) summarizes organoid composition after having been formed in media comprising different WNT signaling agonists or antagonists. Bars represent mean of 2 experiments +/−standard deviation, as normalized to a control lacking exogenously-added WNT signaling modulators.

FIG. 12 shows the effects of modulating signaling through various ERBB receptor family members on organoids formed from non-sorted human mammary epithelial cells. The flow cytometry plots in panel A) show a comparison of organoid composition after having been formed in an organoid medium comprising either no modulation of ERBB receptor family members or the indicated ERBB receptor family ligands. The flow cytometry plots in panel B) show, for two different donors, the effects on organoid formation of organoid media either excluding exogenous modulators of ERBB family members or comprising inhibitors of EGF signaling.

FIG. 13 shows the effects of modulating signaling through ERBB family members on organoids formed from non-sorted human mammary epithelial cells. The plot in panel A) shows the total number of cells included among organoids formed in an organoid medium including either an ERBB agonist (AREG, EGF, or NRG1) or antagonists (Erlotinib+Gefitinib). The bar graph in panel B) summarizes the resulting organoid composition after having been formed in an organoid media comprising different ERBB receptor modulators. Bars represent mean of 2 experiments +/−standard deviation, as normalized to a control lacking exogenously-added ERBB family signaling modulators.

FIG. 14 shows the effects of modulating signaling through TGFβ on organoids formed from non-sorted human mammary epithelial cells. The flow cytometry plots in panel A) show a comparison of organoid cellular composition after having been formed in an organoid medium including either no modulation of TGFβ signaling or including exogenously-added TGFβ1. The low cytometry plots in panel B) show the effects of exogenously-added inhibitors of TGFβ signaling on organoid formation.

FIG. 15 shows the effects of inhibiting signaling through TGFβ on ER localization. In panel A) (human) and in panel B) (mouse) organoids were formed in respective organoid media of this disclosure, and were then transiently exposed to different inhibitors of TGFβ signaling. Organoids were imaged by confocal microscopy after staining for K8, K14, ER and the nuclear stain DAPI. Arrowheads show the co-localization of ER and DAPI staining.

FIG. 16 shows representative images of passage 1, day 10 organoids formed from non-sorted human mammary epithelial cells. The organoids were formed in an organoid medium comprising no exogenously added RSPO-1, Noggin, progesterone, or EGF. The image in panel A) shows a wider focal area capturing a plurality of formed organoids. Scale bar is 100 μm. The images in panel B)-D) show a single formed organoid imaged by confocal microscopy after staining for K8, K14, and the nuclear stain DAPI, either alone or merged. Scale bar is 50 μm.

FIG. 17 shows representative confocal microscopy images of organoids formed from non-sorted human mammary epithelial cells. Luminal-restricted organoids were formed in an organoid medium including either 50 ng/mL Neuregulin 3 (NRG3) (A) or 100 ng/mL anti mullerian hormone (AMH) (B). Basal-restricted organoids were formed in an organoid medium including 50 ng/mL of Granulocyte-macrophage Colony Stimulating Factor (GM-CSF) (C). Organoids were stained for K8, K14, and the nuclear stain DAPI. Scale bars are 100 μm.

### DETAILED DESCRIPTION

This disclosure relates to media and methods for growing mammary organoids. More specifically, this disclosure relates to manipulating a culture of mammary epithelial cells to predictably and reproducibly yield mammary organoids composed of desired mammary gland cell types. In some embodiments, mammary organoids formed in a medium of this disclosure or by practicing methods of this disclosure may be expanded or passaged in the medium.

Where used in this disclosure, the term “mammary organoid” or “mammary organoids” refers to a multicellular structure that recapitulates the general organization of the mammary epithelium, or a specific component thereof, such as a segment of mammary duct or a terminal duct lobular unit. In one embodiment, a mammary organoid is composed of more of a specific cell type (e.g. luminal cells) than of any other types of cells (e.g. basal, endothelial, stromal, hematopoietic, etc). In a related embodiment, such a mammary organoid may be composed of mostly, or completely, a single type of mammary epithelial cell. In one embodiment, a mammary organoid is composed of a more balanced mixture of various types of mammary epithelial cells.

As an example of the former, a “luminal organoid”, a “luminal-restricted organoid”, or “luminal-biased organoid” is a mammary organoid (formed using organoid media of this disclosure) that is composed of about 50%, about 60%, about 70%, about 80%, about 90% or more luminal cells. Thus, a luminal organoid may be composed of more luminal cells than non-luminal cells. Where formed using organoid media of this disclosure, such luminal organoids may be referred to as a first population of organoids. In one embodiment, a luminal organoid (or a first population of organoids) is composed of more luminal cells than would otherwise be present if using media formulated differently from organoid media, as disclosed herein.

As an example of the latter, a “mixed lineage organoid” or “a branched organoid” is a mammary organoid composed of various cell types (e.g. basal cells, luminal cells, stromal cells, etc). A mixed lineage organoid may be organized substantially as observed in normal mammary tissue. Where formed using media formulated differently from organoid media of this disclosure, such mixed lineage organoids may be referred to as a second population of organoids. Thus, organoids of a second population of organoids are composed of at least more basal cells and fewer luminal cells then had they been exposed to an organoid medium of this disclosure. A mixed lineage organoid may be formed using a modified organoid medium of this disclosure, whether beginning from isolated mammary epithelial cells or from a first population of organoids.

Where used in this disclosure, the term “mammary epithelial cells” refers to those cells present in and isolatable from the mammary glands of mammals. In one embodiment, the mammary epithelial cells may comprise mammary epithelial stem or progenitor cells. For the purposes of this disclosure, the mammary epithelial cells may be provided as a single cell suspension, a suspension of fragments of mammary epithelial cells, as clumps of mammary epithelial cells, or a mixture of any combination of the foregoing. In one embodiment, the mammary epithelial cells may be “mammary epithelial-like cells” if derived from pluripotent stem cells, such as induced pluripotent stem cells, embryonic stem cells, or the like. In one embodiment, the mammary epithelial cells or the mammary epithelial-like cells originate from a primate, such as a human, or a rodent, such as a mouse.

Mammary epithelial cells typically comprise basal cells (for example, cells characterized by one or more of: K14⁺, K5⁺, SMA⁺, CD49f⁺, and K8⁻) and luminal cells, the luminal cells further subdivided into an ER⁺ lineage (for example, cells characterized by one or more of: K8⁺, ER⁺, K5⁻, K14⁻, and CD49f⁻) and a milk producing lineage (for example, cells characterized by one or more of: K8⁺, CD49b⁺, ALDEFLUOR⁺). In one embodiment, luminal cells may be categorized on the basis of K8 expression (“K8⁺”). In one embodiment, luminal cells may be categorized on the basis of K8 and K18 co-expression. In one embodiment, basal cells may be categorized on the basis of K14 expression (“K14⁺”). In one embodiment, luminal cells may be categorized on the basis of K14 and K5 co-expression. The skilled person will know that the signatures of basal or luminal cells is neither exhaustive nor exclusive. In some cases different markers may help distinguish the respective cell types. Or, in some cases different cell types may express common markers, either at more or less equivalent levels or at different levels.

Basal and luminal cells are likely bipotent insofar that a sorted population of luminal cells, for example, may be cultured under conditions that promote the eventual emergence of basal cells, and vice versa, provided that an appropriate culture environment is used. In one embodiment, the appropriate culture environment includes any appropriately supplement culture medium (as further described herein).

A preparation of mammalian epithelial cells may be obtained using known methodologies or variations of known methodologies. Typically, mammary glands are resected from subjects and physically/mechanically disrupted using a scalpel, or other like means. Enzymatic digestion will usually facilitate the further dissociation of the mammary gland, such as connective tissues thereof.

In one embodiment, mammary tissue minced using a scalpel may be gently agitated in a solution including collagenase and hyaluronidase to break down extracellular matrix and connective tissue. The arising liquid fraction may then be subjected to a single or multiple rounds of centrifugation.

If performing multiple rounds of centrifugation, each round may be done at incrementally higher centrifugal force and/or longer duration, saving the pellet at each step. In one embodiment, two to three rounds of centrifugation are sufficient to isolate cells of interest. For example, after a first centrifugation at approximately 100×g for about 0.5-1 minute, larger debris including fragments of mammary epithelial cells may be recovered in the pellet. After a second centrifugation at approximately 200×g for about 3-5 minutes, clumps of and individual mammary epithelial cells may be recovered in the pellet. And, after a third centrifugation at approximately 400×g for about 5 minutes, individual cells including stromal cells may be recovered in the pellet.

In some embodiments it may be necessary, depending on the appearance of the pelleted cells, to briefly treat with ammonium chloride to lyse contaminating red blood cells.

For the purpose of generating a single cell suspension of mammary epithelial cells, the cells of the first, and if applicable the second, pellet may be further processed by sequential treatment with a trypsin-containing solution and then a DNase solution, optionally followed by filtration through a 37 μm filter.

In some embodiments, the cultured mammary epithelial cells may be comprised in relatively larger tissue fragments, such as those obtained as a residue after digestion and filtration as described above.

In some embodiments, the cultured mammary epithelial cells may be single cells obtained using a process as described above.

In some embodiments, the cultured mammary epithelial cells may be a mixture of single cells and tissue fragments, whether smaller (e.g. those that pass through a 37 μm filter) and/or larger (e.g. those that do not pass through a 37 μm filter).

However processed, mammary epithelial cells may be seeded and cultured in media (e.g. organoid media) as disclosed herein and/or in accordance with the methods disclosed herein.

### Media

In one aspect of this disclosure are provided cell culture media (i.e. organoid media) for forming or growing mammary organoids. In one embodiment, organoid media of this disclosure maintain and/or expand mammary organoids across a number of passages. In one embodiment, organoid media may be used to grow/form mammary organoids from mammary epithelial cells and to maintain/expand mammary organoids across a number of passages.

Where used in this disclosure, the term “organoid medium” refers to a solution that may be used to form and/or grow and/or expand mammary organoids from an isolated preparation of mammary epithelial cells. Furthermore, such an organoid medium may also be used to passage arising mammary organoids formed from an isolated preparation of mammary epithelial cells. In one embodiment, an organoid medium comprises a basal medium and is appropriately supplemented with additives for the culture of mammalian cells, particularly primary epithelial cells, such as: one or more of salt(s); buffer(s); amino acids; energy source(s) (e.g. glucose, pyruvate, etc); albumin(s) or albumin surrogates; trace elements; and lipids. Additionally, an organoid medium may comprise one or more appropriate small molecules and/or cytokines or growth factors, or mimetics thereof, to bias the formation of mammary organoids (from mammary epithelial cells) composed of cells of a desired lineage. In one embodiment, such an organoid medium may also promote growth/expansion of the mammary organoids. In a specific embodiment, organoid media are intended for forming and/or growing and/or expanding luminal organoids, as may be comprised in a first population of organoids. In one embodiment, the same organoid media may be used to form/passage human and mouse luminal organoids. In one embodiment, different organoid media may be used to form/passage human and mouse luminal organoids.

Organoid media may be formulated using any known and/or commercially available basal medium capable of supporting a culture of epithelial cells, such as mammary epithelial cells. For example, basal media routinely used in the culture of epithelial cells, such as mammary epithelial cells, include DMEM, DMEM/F12, Adv-DMEM, and Adv-DMEM/F12, and the like. In one embodiment, the basal medium is either DMEM, DMEM/F12, Adv-DMEM, or Adv-DMEM/F12.

On the one hand, if it is desired to bias the culture of mammary epithelial cells to form luminal organoids composed of more luminal cells than might otherwise occur, then a formulation of an organoid medium may need to be optimized for this particular purpose.

In one embodiment, organoid media include at least one mitogen. The at least one mitogen may be based on an amino acid sequence corresponding to a human gene. In one embodiment, the mitogen may be based on an amino acid sequence corresponding to a non-human gene, such as a rodent species. In one embodiment, it is appropriate to match the origin of the mitogen (in terms of sequence or sourcing) with the species origin of the isolated mammary epithelial cells to be cultured. In some embodiments, it is not necessary to match the origin of the mitogen (in terms of sequence or sourcing) with the species origin of the isolated mammary epithelial cells to be cultured.

In one embodiment, organoid media (for forming and maintaining luminal organoids) include a ligand of ERBB1. In one embodiment, the ligand of ERBB1 is not epidermal growth factor (EGF). In the same or different embodiment, the ligand of ERBB1 is not transforming growth factor alpha (TGFalpha). Thus, in one embodiment, the ligand of ERBB1 is neither EGF nor TGFalpha, or a functional fragment or mimetic of the foregoing.

In one embodiment, the ligand of ERBB1 is amphiregulin. In such an embodiment, the concentration of amphiregulin may be between about 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL.

In one embodiment, organoid media (for forming and maintaining luminal organoids) include a ligand of ERBB4. In one embodiment, the ligand of ERBB4 is also a ligand of a different ERBB receptor family member. In such an embodiment, the ligand of ERBB4 may also be a ligand of ERBB3.

In one embodiment, the ligand of ERBB4 is heregulin (aka Neuregulin 1, a ligand of dimerized ERBB3 and ERBB4). In such an embodiment, the concentration of heregulin is between about 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL. In one embodiment, the ligand of ERBB4 is Neuregulin 3, and it may be included in organoid media within the foregoing concentration ranges.

In one embodiment, organoid media include one of a ligand of ERBB1 or ERBB4. In one embodiment, organoid media comprise both a ligand of ERBB1 and ERBB4.

In one embodiment, the ligand of ERBB4 may be Betacellulin, Epigen, Epiregulin, Neuregulin 1, Neuregulin 2, Neuregulin 3, Neuregulin 4, or Tomoregulin. In some embodiments, the organoid medium (for promoting and maintaining basal or mixed organoids) comprises more than one ERBB4 ligand.

In one embodiment, the ligand of ERBB4 is Neuregulin 3. In such an embodiment, the concentration of Neuregulin 3 is between about 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL. In one embodiment, the concentration of Neuregulin 3 is about 50 ng/mL.

As important as it may be to activate and/or inhibit certain signaling cascades using protein, peptide, or small molecule ligands, it may also be important to not activate and/or not inhibit other signaling cascades.

Accordingly, in one embodiment, organoid media (for forming and maintaining luminal organoids) do not include one or both of an exogenously-added WNT signaling agonist and a modulator of BMP signaling (such as an activator or an inhibitor).

In one embodiment, the WNT signaling agonist not to be included in an organoid medium is an R-spondin, a WNT protein, or an engineered mimetic of either of the foregoing. Whereas WNT signaling agonists are used in the culture of certain epithelial organoids, the inventors have shown that inclusion of a WNT signaling agonist in an organoid medium of this disclosure may be dispensable or detrimental for forming, promoting growth of, and maintaining luminal organoids. Inclusion of a WNT signaling agonist in an organoid medium, may also promote/maintain the formation/growth of mixed lineage organoids.

In one embodiment, the R-spondin excluded from an organoid medium is one or more of R-spondin 1, R-spondin 2, R-spondin 3, or R-spondin 4. In one embodiment, the R-spondin excluded from the organoid medium is each of R-spondin 1, R-spondin 2, R-spondin 3, or R-spondin 4. Accordingly, the concentration of an R-spondin added to an organoid medium for promoting and maintaining luminal organoids is 0 ng/mL, or effectively 0 ng/mL.

In one embodiment, the WNT protein excluded from an organoid medium is one or more of WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, or WNT16. In another embodiment, the WNT protein excluded from the organoid medium is each of WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, or WNT16. Accordingly, the concentration of a WNT protein added to an organoid medium for promoting and maintaining luminal organoids is 0 ng/mL, or effectively 0 ng/mL.

Advances in synthetic biology have enabled the in silico design of ligand mimetics, whether protein-based, peptide-based or small molecule-based. Advantages of such mimetics may include, but are not limited to, increased activity, increased stability, simplified structure, decreased size, etc. Thus, in some embodiments, WNT or R-spondin function, such as via a mimetic thereof, is absent from an organoid medium of this disclosure.

By way of example, typical recombinant WNT proteins have little or no effect when added to culture media because they rapidly degrade, possibly due to a high hydrophobicity. To overcome this challenge a canonical, FZD-mediated, WNT pathway agonist was engineered to be more potent than wild-type Wnt3a while retaining acceptable biological activity when added to culture media (U-Protein Express).

In one embodiment, the modulator of BMP signaling not to be included in an organoid medium is a protein, a peptide, or small molecule that inhibits signaling through a BMP receptor. In one embodiment, the modulator of BMP signaling not to be included in an organoid medium is a protein, a peptide, or small molecule that activates signaling through a BMP receptor. Whereas modulators of BMP signaling are commonly used to culture certain epithelial organoids, the inventors have shown that inclusion of certain modulator(s) of BMP signaling in an organoid medium of this disclosure may be dispensable or detrimental to promoting and maintaining luminal organoids.

In one embodiment, the inhibitor of BMP signaling excluded from an organoid medium (for promoting/maintaining luminal organoids) is a protein, or a peptide. Such excluded protein (or functionally equivalent peptide) may be one or more of Noggin, Chordin, Follistatin, Sclerostin, CTGF/CCN2, gremlin, Cerberus, DAN, PRDC, decorin, alpha-2 macroglobulin proteins, or the like. In one embodiment, the BMP signaling inhibitor excluded from an organoid medium is each of Noggin, Chordin, Follistatin, Sclerostin, CTGF/CCN2, gremlin, Cerberus, DAN, PRDC, decorin, and alpha-2 macroglobulin proteins. Accordingly, the effective concentration of an inhibitor of BMP signaling in an organoid medium for promoting and maintaining luminal organoids is 0 ng/mL, or effectively 0 ng/mL.

In one embodiment, the inhibitor of BMP signaling excluded from an organoid medium (for promoting/maintaining luminal organoids) is a small molecule. Such excluded small molecule may be LDN 193189 or dorsomorphin. In one embodiment, the inhibitor of BMP signaling excluded from an organoid medium is each of LDN 193189 and dorsomorphin. Accordingly, the effective concentration of a BMP signaling inhibitor in an organoid medium for promoting and maintaining luminal organoids is 0 ng/mL, or effectively 0 ng/mL.

In one embodiment, the activator of BMP signaling may be a protein such as BMP2 or BMP4, or a peptide thereof.

Many modulators of BMP signaling are known and it may be important to select specific modulators for inclusion or omission from an organ medium based on the pathways they regulate. In some embodiments of organoid media, optimal formation and maintenance of luminal organoids may be achieved by including two or more different modulators of signaling through BMP. In one embodiment, more than one inhibitor of BMP signaling or more than one activator of BMP signaling may be included in an organoid medium. In one embodiment, both an activator and an inhibitor of BMP signaling may be included in an organoid media. In any embodiment including BMP signaling inhibitors or BMP signaling activators, or combinations of the two, the specific BMP pathways modulated may be an important consideration for forming luminal organoids.

In one embodiment, an organoid medium for forming/maintaining luminal organoids includes both an ERBB1 and ERBB4 ligand and is free of one or both of an exogenously-added WNT signaling agonist and an inhibitor of BMP signaling (or a mimetic of any of the foregoing).

In one embodiment, organoid media comprise an inhibitor of BMP signaling but not an exogenously-added WNT signaling agonist. In one embodiment, organoid media comprise a WNT signaling agonist but not an exogenously-added inhibitor of BMP signaling.

Biasing or promoting the formation of luminal organoids may mean that culturing mammary epithelial cells in an organoid medium enriches organoids composed of more luminal cells than non-luminal cells (for example, as compared to forming organoids from isolated mammary epithelial cells in a medium different from the organoid medium). In one embodiment, luminal organoids formed in an organoid medium comprise greater than 70% luminal cells and about 20% basal cells or less. In one embodiment, luminal organoids formed in an organoid medium comprise greater than 80% luminal cells and about 15% basal cells or less. In one embodiment, luminal organoids formed in an organoid medium comprise about 90% luminal cells or more and about 5% basal cells or less.

On the other hand, if it is desired that formation of luminal organoids (i.e. a first population of organoids) is subverted to rather form, for example, mixed lineage organoids (i.e. a second population of organoids), then a modified organoid medium may be used for this purpose. In one embodiment, a modified organoid medium may comprise one or more of, or two or more of: a mitogen; an agonist of WNT signaling, or a modulator of BMP signaling.

In one embodiment, the modified organoid medium (for forming and maintaining mixed lineage organoids) comprises one or more ligand of one or more of ERBB1, ERBB2, ERBB3, or ERBB4.

In one embodiment, the ligand of ERBB1 may be epidermal growth factor (EGF), transforming growth factor alpha (TGFalpha), amphiregulin, heparin-binding EGF (HB-EGF), betacellulin, epigen, or epiregulin. In some embodiments, the modified organoid medium may comprise more than one ERBB1 ligand.

In one embodiment, the ligand of ERBB1 is EGF and/or TGFalpha and/or amphiregulin. In such an embodiment, the concentration of such ligand(s) of ERBB1 may be between about 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL.

In one embodiment, a ligand of ERBB3 included in a modified organoid medium may be one or more of Neuregulin 1, Neuregulin 2, or Neuroglycan C. In some embodiments, the modified organoid medium (for forming and maintaining mixed lineage organoids) may comprise more than one ERBB3 ligand.

In one embodiment, the ligand of ERBB3 is Neuregulin 1. In such an embodiment, the concentration of Neuregulin 1 is between about 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL

In one embodiment, the WNT signaling agonist included in a modified organoid medium, is an R-spondin, a WNT protein, or an engineered mimetic of either of the foregoing. Inclusion of a WNT signaling agonist in a modified organoid medium, alone or in combination with other factors, may subvert the formation/maintenance of luminal organoids and bias a culture of mammary epithelial cells to mixed lineage organoids (e.g. a second population of organoids).

In one embodiment, the R-spondin included in a modified organoid medium is one or more of R-spondin 1, R-spondin 2, R-spondin 3, or R-spondin 4. In one embodiment, the R-spondin included in the modified organoid medium is each of R-spondin 1, R-spondin 2, R-spondin 3, or R-spondin 4. Accordingly, the effective concentration of an R-spondin in a modified organoid medium for forming and maintaining mixed lineage organoids is between 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL.

In one embodiment, the WNT protein included in a modified organoid medium is one or more of WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, or WNT16. Accordingly, the effective concentration of a WNT protein in a modified organoid medium for forming and maintaining mixed lineage organoids is between 1 μg/mL to 0.1 ng/mL, or between about 500 ng/mL to 0.5 ng/mL, or between about 250 ng/mL to 1 ng/mL, or between about 125 ng/mL to 2 ng/mL, or between about 50 ng/mL to 3 ng/mL.

As Wnt3a may be unstable in cell culture medium or when used under cell culture conditions, in some embodiments Wnt3a-conditioned medium can be an appropriate substitute for Wnt3a in a modified organoid medium of this disclosure. WNT-conditioned medium can be generated using known approaches such as through the culture of WNT-producing cells. The concentration of WNT in a WNT-conditioned medium is typically commensurate with the concentrations outlined above.

Advances in synthetic biology have enabled the in silico design of ligand mimetics. Advantages of such mimetics may include, but are not limited to, increased activity, increased stability, simplified structure, decreased size, etc. Thus, in some embodiments, WNT or R-spondin function via a mimetic thereof, is present in an organoid medium of this disclosure.

By way of example, typical recombinant WNT proteins have little or no effect when added to culture media because they rapidly degrade, possibly due to a high hydrophobicity. To overcome this challenge a canonical, FZD-mediated, WNT pathway agonist was engineered to be more potent than wild-type Wnt3a while retaining acceptable biological activity when added to culture media (U-Protein Express).

In one embodiment, a WNT mimetic is included in a modified organoid medium for subverting formation/maintenance of luminal organoids while promoting formation/maintenance of mixed lineage organoids. In one embodiment, a WNT mimetic may be included in such medium alone or together with one or more other agonists of WNT signaling. In embodiments, where a WNT mimetic is included in such medium, the WNT mimetic may be WNT Surrogate-FC Fusion Protein. If included in a modified organoid medium for subverting formation/maintenance of luminal organoids, while promoting formation/maintenance of basal or mixed organoids, the concentration of the WNT mimetic may be between about 100 nM and 0 nM, between about 50 nM and 0.01 nM, between about 20 nM and 0.05 nM, or between about 10 nM and 0.1 nM.

In one embodiment, a modulator of BMP signaling included in a modified organoid medium for subverting formation/maintenance of luminal organoids, while promoting formation/maintenance mixed lineage organoids, is a protein, a peptide, or small molecule. In one embodiment, such modulator may be an activator or an inhibitor of BMP signaling.

In embodiments where the modified organoid medium comprises an activator of BMP signaling, examples of such activators include BMP2 or BMP4, or the like. Accordingly, the effective concentration of an activator of BMP signaling in a modified organoid medium may be between about 10 μg/mL to 0.1 ng/mL, or about 1 μg/mL to 1 ng/mL, or about 300 ng/mL to 3 ng/mL, or about 150 ng/mL to 5 ng/mL, or about 100 ng/mL to 10 ng/mL, or about 50 ng/mL to 10 ng/mL.

In embodiments where the modified organoid medium comprises an inhibitor of BMP signaling, examples of such protein (or peptide-based) inhibitors include Noggin, Chordin, Follistatin, Sclerostin, CTGF/CCN2, gremlin, Cerberus, DAN, PRDC, decorin, alpha-2 macroglobulin proteins, or the like. Accordingly, the effective concentration of an inhibitor of BMP signaling in a modified organoid medium may be between about 10 μg/mL to 0.1 ng/mL, or about 1 μg/mL to 1 ng/mL, or about 300 ng/mL to 3 ng/mL, or about 150 ng/mL to 5 ng/mL, or about 100 ng/mL to 10 ng/mL, or about 50 ng/mL to 10 ng/mL.

In embodiments where the modified organoid medium comprises an inhibitor of BMP signaling, examples of such small molecule inhibitors include LDN 193189 or dorsomorphin. Such an inhibitor of BMP signaling included in a modified organoid medium may be between about 1 mM to 0.001 nM, about 1 μM to 0.1 nM, about 100 nM to 1 nM.

In one embodiment, an organoid medium may include an inhibitor of BMP signaling, whether protein/peptide-based or a small molecule (or a combination of the two), thus a modified organoid medium may include an activator or an agonist of BMP signaling, or vice versa.

Thus, in one embodiment, a modified organoid medium for subverting formation/maintenance of luminal organoids, while promoting formation/maintenance of mixed lineage organoids, includes two or more of: at least one ERBB ligand; at least one agonist of WNT signaling; and at least one modulator of BMP signaling.

In another aspect of this disclosure are provided kits comprising components for forming mammary organoids. In one embodiment a different kit is used to form mouse mammary organoids than is used to form human mammary organoids. The description above with regard to media compositions is incorporated into the following description of kits.

Kits of this disclosure will comprise a basal medium and at least one supplement to be added to the basal medium. In one embodiment, kits of this disclosure comprise a basal medium and at least two supplements to be added to the basal medium. In one embodiment, kits of this disclosure comprise a basal medium and three or more supplements to be added to the basal medium.

Any particular supplement may be combined with the basal medium if it is desired to form a specific type of mammary organoid. For example, if it is desired to form a first population of organoids (e.g. luminal-restricted or luminal-biased organoids) then a first supplement to be added to the basal medium may comprise one of or both of a ligand of ERBB1 and a ligand of ERBB4. In some embodiments, the first supplement is also free of one of or both an exogenously-added WNT signaling agonist and a modulator of BMP signaling. In such embodiments, the modulator of BMP signaling may be an activator or inhibitor of BMP signaling. In a related embodiment, the first supplement comprises one of or both of a ligand of ERBB1 and a ligand of ERBB4, and a modulator of BMP signaling, and is free of a WNT signaling agonist. In a related embodiment, the first supplement comprises one of or both of a ligand of ERBB1 and a ligand of ERBB4, and a WNT signaling agonist, and is free of a modulator of BMP signaling. In a related embodiment, the first supplement comprises one of or both of a ligand of ERBB1 and a ligand of ERBB4, and is free of both a WNT signaling agonist and a modulator of BMP signaling. In one embodiment, the ligand of ERBB1 is not EGF. In one embodiment, neither the basal medium nor the first supplement comprise progesterone.

Kits may further comprise a second supplement to be added to the basal medium for subverting a formed first population of organoids (e.g. luminal-restricted or luminal-biased organoids) to a second population of organoids (e.g. mixed organoids). A basal medium supplemented with the second supplement (and optionally also supplemented the first supplement) has been referred to herein as a modified organoid medium. In embodiments of the second supplement, it may comprise one of or both of a WNT signaling agonist and a BMP signaling modulator. In such embodiments, the modulator of BMP signaling may be an activator or inhibitor of BMP signaling. In a related embodiment, the BMP signaling modulator comprised in the second supplement is an activator of BMP signaling, such as BMP2 or BMP4. In a related embodiment, the second supplement may further comprise a ligand of ERBB1 that is different from the ligand of ERBB1 comprised in the first supplement. In one embodiment, the ligand of ERBB1 comprised in the second supplement is EGF.

Kits may further comprise a third supplement to be added to the basal medium (whether or not in the presence of one of or both of the first and second supplements). The third supplement may comprise an inhibitor of TGFβ signaling. In one embodiment, the third supplement is added to the basal medium (or to the organoid medium or to the modified organoid medium) after the mammary organoids of interest have been formed to promote the nuclear localization of estrogen receptor. In one embodiment, the inhibitor of TGFβ signaling is SB431542. In one embodiment, the inhibitor of TGFβ signaling is RepSox.

Any of the media described above (including embodiments comprised in kits) may be used in methods to form and/or maintain mammary organoids, whereby the type of mammary organoid that may be formed and/or maintained is dependent on the formulation of the medium (e.g. organoid media vs modified organoid media). Since mammary epithelial cells, and the organoids arising therefrom, are sex hormone responsive, in some embodiments it may be desirable that media (e.g basal media, organoid media, and modified organoid media) and supplements added thereto of this disclosure, whether used in methods or bundled into kits, are free of an exogenously added sex hormone. In one embodiment, the sex hormone is estrogen. In one embodiment, the sex hormone is progesterone.

### Methods

In another aspect of this disclosure are provided methods for forming or growing mammary organoids. In one embodiment, the methods of this disclosure maintain and/or expand mammary organoids across a number of passages. The mammary organoids formed/expanded/passaged in accordance with the disclosed methods may be used in downstream assays, such as to better understand mammary gland biology in both normal and diseased states, in drug screening and toxicity studies, or in therapeutic applications.

The methods of forming mammary organoids, and specifically a first population of organoids, from isolated mammary epithelial cells comprise contacting mammary epithelial cells with an organoid medium free of one or both of an exogenously-added WNT signaling agonist and/or a modulator of BMP signaling. In such embodiments the modulator of BMP signaling may be an inhibitor of BMP signaling, or it may be an activator of BMP signaling.

In one embodiment, the methods use organoid medium comprising a modulator of BMP signaling (e.g. the inhibitor or activator of BMP signaling) but not a WNT signaling agonist. In one embodiment, the methods use organoid medium comprising a WNT signaling agonist but not a modulator of BMP signaling (e.g. the inhibitor or activator of BMP signaling). In one embodiment, the methods use organoid medium comprising neither a modulator of BMP signaling (e.g. the inhibitor or activator of BMP signaling) nor a WNT signaling agonist.

In one embodiment, the mammary epithelial cells comprise mammary epithelial stem or progenitor cells.

The isolated mammary epithelial cells may be of any mammalian species. In one embodiment, the mammary epithelial cells are either human mammary epithelial cells or mouse mammary epithelial cells. Isolated mammary epithelial cells may be obtained from a commercial supplier, an academic collaborator, or by processing mammary tissue. If starting from mammary tissue, such specimen is usually processed using a combination of mechanical/physical and enzymatic means.

As described herein, a single cell suspension of mammary epithelial cells or a suspension of fragments or clumps of mammary epithelial cells is typically generated by cutting the mammary tissue using a scalpel, a tissue dissociator, or the like. Following or during the cutting operation, the mammary tissue is typically incubated in an enzyme-containing solution to breakdown extracellular matrix and connective tissue. The enzyme solution could include any enzyme or combination of enzymes useful for the purpose of processing the mammary tissue. By way of non-limiting example, enzymes that may be used for this purpose include collagenase, hyaluronidase, dispase, thermolysin, trypsin, DNase, etc.

In one embodiment, the enzyme treatment of minced mammary tissue is applied sequentially. For example, minced mammary tissue may first be incubated in a solution including one or both collagenase and hyaluronidase. Larger fragments of the digested mammary tissue may be collected by centrifugation at a relatively low centrifugal force (e.g. <200×g) and set aside. To isolate relatively smaller material, including clumps of cells or single cells, the resulting supernatant can be further centrifuged at an incrementally higher centrifugal force (e.g. >200×g). Both pellets can be combined and further digested in a trypsin-containing solution to obtain a suspension of substantially single cells.

In some embodiments the suspension of substantially single cells, or fragments, or a mixture of single cells and fragments may require DNase treatment to degrade DNA from lysed cells and reduce the viscosity of the suspension of cells.

After generating a suspension of mammary epithelial cells, particularly where single cells are comprised therein, it may be desirable to separate a specific subset of mammary epithelial cells from a background of various cell types in the sample. A specific subset of mammary epithelial cells may be separated from the sample using known methods such as by fluorescence activated cell sorting or immunomagnetic cell separation, as commercialized by STEMCELL Technologies.

The cellular composition of the mammary gland includes both epithelial and non-epithelial (fibroblasts, endothelial cells, lymphocytes, adipocytes, neurons and myocytes) cells. The expression of markers between corresponding cell types may vary between humans and mice. In humans, epithelial cells may be distinguished from non-epithelial cells by the expression of EpCAM. Within the EpCAM⁺ epithelial compartment, the mammary epithelium includes two main lineages: the luminal lineage and the myoepithelial (or basal) lineage. On the one hand, myoepithelial cells are typically marked by: i) high expression of cytokeratin 5 (K5), cytokeratin 14 (K14), P63, Smooth Muscle Actin (SMA), CD49f, and CD29; and ii) the absence of cytokeratin 8 (K8), cytokeratin (K18) and CD24. On the other hand, the luminal lineage can be divided into 2 subpopulations: milk progenitor cells and a hormone-responsive ER⁺ lineage. The ER⁺ lineage may be distinguished by: i) high expression of K8, K18, Estrogen Receptor-alpha (ERa), Progesterone receptor (PR); and ii) an absence of K5, K14, CD49f and SMA. The milk lineage may be distinguished by: i) positive ALDEFLUOR staining, indicative of ALDH1A3 expression; ii) expression of K8, K18, and CD49f; iii) expression of K5, and K14; and (iv) an absence of ERα and SMA. In mice, epithelial cells may be distinguished from non-epithelial cells by the expression of EpCAM or CD24. Within the EpCAM⁺ epithelial compartment, the mammary epithelium includes two main lineages: the luminal lineage and the myoepithelial (or basal) lineage. On the one hand, myoepithelial cells are typically marked by: i) high expression of cytokeratin 5 (K5), cytokeratin 14 (K14), P63, Smooth Muscle Actin (SMA), CD49f, and CD29; and ii) the absence of cytokeratin 8 (K8), cytokeratin (K18). On the other hand, the luminal lineage can be divided into 2 subpopulations: milk progenitor cells and a hormone-responsive ER⁺ lineage. The ER⁺ lineage may be distinguished by: i) high expression of K8, K18, Estrogen Receptor-alpha (ERα), Progesterone receptor (PR); and ii) an absence of K5, K14, CD49f, CD49b and SMA. The milk lineage may be distinguished by: i) positive ALDEFLUOR staining, indicative of ALDH1A3 expression; ii) expression of K8, K18, CD49b, and CD49f; iii) low expression of K5, K14, ERα, and SMA. In some cases presence or absence of one or both of SMA and ER+ may help to further distinguish basal and luminal cells.

In some embodiments, isolated mammary epithelial cells are cultured in association with an extracellular matrix. In one embodiment, the isolated mammary epithelial cells are seeded within a “dome” of an extracellular matrix. In one embodiment, the isolated mammary epithelial cells are seeded within a “sandwich” of an extracellular matrix, wherein the cells are seeded onto a layer of the extracellular matrix and then covered by an overlay of the same or a different extracellular matrix. In one embodiment, the isolated mammary epithelial cells are seeded on top of a layer of an extracellular matrix.

Extracellular matrix for culturing mammary epithelial cells are known, and any appropriate extracellular matrix is contemplated in this disclosure. In one embodiment, the extracellular matrix is Matrigel™ (Corning). In one embodiment, the extracellular matrix may be a suitable replacement for Matrigel, such as Cultrex™ Basement Membrane Matrix (Trevigen).

In one embodiment, the extracellular matrix may be a single natural component of extracellular matrix, or any combination thereof. Examples of natural components of extracellular matrix include collagen, laminin, entactin, heparin sulfate, proteoglycans, or fibronectin.

In one embodiment, the extracellular matrix may be a synthetic matrix, such as a hydrogel formulated with any one or more of the natural extracellular components outlined above.

The density of mammary epithelial cells seeded on a layer an extracellular matrix or within a specified volume of an extracellular matrix should be empirically determined. In embodiments where mammary epithelial cells are seeded within a “dome” of extracellular matrix at the bottom of a 24-well plate, anywhere from about 1,000 to 50,000 cells can be included within a 30-50 μL dome. When using different plate formats the cell number and volume of extracellular matrix may require adjustment. Nevertheless, it should be appreciated that down to a single cell or clonal densities of cells can be seeded on or in an extracellular matrix.

As described hereinabove, the nature of the organoid medium will depend on the intended output of the methods disclosed herein. Thus, the description of organoid media hereinabove are incorporated into the following disclosures relating to methods of forming mammary organoids.

If the desired output of the methods is a first population of organoids (e.g. luminal organoids), then the organoid medium may comprise a basal medium and one or both of a ligand of ERBB1 and/or a ligand of ERBB4 (along with being free of one or both of an exogenously added WNT signaling agonist and/or a modulator (e.g. inhibitor or activator) of BMP signaling).

Potential ligands of ERBB1 and ERBB4 that may be comprised in an organoid medium are described hereinabove. In one embodiment, the ligand of ERBB1 is amphiregulin. In one embodiment, the ligand of ERBB4 is neuregulin 1. In one embodiment of an organoid medium for promoting the formation and/or maintenance of luminal organoids, the medium includes both a ligand of ERBB1 and a ligand of ERBB4. In one embodiment, the ligand of ERBB1 is not EGF or TGFalpha. In one embodiment, the ligand of ERBB1 is neither EGF nor TGFalpha.

Also as described above, an important consideration when formulating an organoid medium for promoting the formation and/or maintenance of luminal organoids is to not activate or to not inhibit certain signaling cascades. Accordingly, in one embodiment of such an organoid medium, the medium is free of one or both of an exogenously-added WNT signaling agonist and/or a modulator of BMP signaling (e.g. an inhibitor or an activator thereof).

In one embodiment, the agonist of WNT signaling not to be included in the organoid medium (for promoting the formation and/or maintenance of luminal organoids) is an R-spondin, a WNT protein or peptide, or an engineered/synthesized mimetic of either of the foregoing.

In one embodiment, the inhibitor of BMP signaling not to be included in the organoid medium (for promoting the formation and/or maintenance of luminal organoids) is a protein inhibitor, such as Noggin, or a small molecule inhibitor, such as LDN193189 or dorsomorphin. In one embodiment, neither of a protein inhibitor of BMP signaling nor a small molecule inhibitor of BMP signaling is including in the organoid medium (for promoting the formation and/or maintenance of luminal organoids).

Thus, the methods further comprise culturing the mammary epithelial cells in an organoid medium for a time sufficient to form a first population of organoids enriched for organoids composed of more luminal cells then non-luminal cells. In other words, the mammary epithelial cells may be cultured in an organoid medium for a time sufficient to form a first population of organoids, wherein organoids formed using a medium different from the organoid medium may yield no organoids at all or organoids composed of fewer luminal cells (and more non-luminal cells) then had an organoid medium of this disclosure been used.

In one embodiment, the luminal organoids may form after about 3 days of being contacted by the organoid medium. In one embodiment, the luminal organoids may form after about 5 days of being contacted by the organoid medium. In one embodiment, the luminal organoids may form after about 7 days of being contacted by the organoid medium. In one embodiment, the luminal organoids may form after about 10 days of being contacted by the organoid medium. In one embodiment, the luminal organoids may form after about 14 days of being contacted by the organoid medium. In one embodiment, the formed luminal organoids may be cultured for up to 30 days or more before needing to be passaged.

In one embodiment, the luminal organoids (i.e. the first population of organoids) formed on contacting mammary epithelial cells with an organoid medium may be passaged 3 or more times, or 5 or more times, or 7 or more times, or 10 or more times.

If the output of the methods is a population of mammary organoids enriched for r mixed lineage organoids (e.g. a second population of organoids), then a modified organoid medium (as described above) may be used to contact and culture the mammary epithelial cells or the first population or organoids. Thus such modified organoid medium may subvert formation of the first population of organoids into a second population of organoids.

Examples of ERBB ligands, agonists of WNT signaling, and modulators of BMP signaling (whether activators or inhibitors) are as specified hereinabove. In one embodiment, a modified organoid medium (for subverting formation of luminal organoids and promoting formation/passaging of mixed lineage organoids) includes each of: at least one ERBB ligand; at least one agonist of WNT signaling; and at least one modulator of BMP signaling.

In one embodiment, a first population of organoids (e.g luminal organoids) formed using organoid media of this disclosure or by practicing methods of this disclosure may be converted to a second population of organoids (e.g. mixed lineage organoids). Converting a first population of organoids to a second population of organoids may be done using a modified organoid medium of this disclosure.

In one embodiment, the mixed lineage organoids (i.e. the second population of organoids) formed on contacting mammary epithelial cells or the first population of organoids with a modified organoid medium may be passaged 3 or more times, or 5 or more times, or 7 or more times, or 10 or more times.

In one embodiment, the methods may further comprise contacting the first population of organoids or the second population of organoids with an inhibitor of TGFβ signaling. Inhibitors of TGFβ signaling are known and commercially available, thus the inhibitor of TGFβ signaling may be any such inhibitor. In one embodiment, the inhibitor of TGFβ signaling is SB431542. In one embodiment, the inhibitor of TGFβ signaling is A77-01. In one embodiment, the inhibitor of TGFβ signaling is A83-01. In one embodiment, the inhibitor of TGFβ signaling is RepSox. Contacting the first population of organoids and/or the second population of organoids with an inhibitor of TGFβ signaling may promote the nuclear localization of estrogen receptor. In one embodiment, contacting the first population of organoids and/or the second population of organoids with an inhibitor of TGFβ signaling is for the duration of the culturing step (in the organoid medium or the modified organoid medium). In one embodiment, contacting the first population of organoids and/or the second population of organoids with an inhibitor of TGFβ signaling is transient, such as after formation of the first population of organoids and/or the second population of organoids.

Regardless of the type of mammary organoid formed by practicing the methods disclosed herein, the organoids may be used in any number of downstream assays, methods, or applications. By way of example, the mammary organoids, whether derived from normal or diseased mammary epithelial cells, may be used to screen a panel of compounds to determine their efficacy and/or toxicity. As another example, such mammary organoids may be used to investigate basic mammary biology or the biology of diseased mammary epithelium, such as cancer. As another example, such mammary organoids may be used to investigate therapeutic interventions wherein they are grafted into a subject, whether a human patient or into an animal model.

The following non-limiting examples are illustrative of the present disclosure.

### EXAMPLES

### Example 1: Processing Mammary Tissue Samples

All cells used in this disclosure were obtained from tissues that were sourced from human or mouse subjects in accordance with applicable IRBs and ethics requirements.

Fragments of human mammary epithelial cells were isolated from tissue samples obtained from academic collaborators, as follows. Resected tissue samples were minced in a petri dish using a scalpel to cut in a cross-hatched pattern. The minced tissue samples were placed in a dissociation flask in standard culture medium supplemented with BSA, insulin, collagenase and hyaluronidase. The flask was gently shaken overnight on an orbital shaker placed in a 37° C. tissue incubator. The following morning the floating layer of fat was removed by pipette, and the remaining liquid was transferred to a tube and briefly centrifuged at low rpm (˜80 g for 30 seconds). The pellet (A pellet) was set aside and the supernatant was further centrifuged for an incrementally longer period of time and at an incrementally higher rpm (˜200 g for 4 minutes). The pellet (B pellet) was set aside and the supernatant was further centrifuged for a still incrementally longer period of time and at an incrementally higher rpm (˜450 g for 5 minutes).

Cryopreserved pellet A and pellet B were dissociated into a single cell suspension by treatment with 0.25% Trypsin-EDTA for 5 minutes at 37° C. (with intermittent trituration) and then centrifuged at (300 g for 5 minutes). The supernatant was discarded and the pellet was resuspended in a 100 μg/ml DNase solution for 1 minute at room temperature. The resulting cells were passed through a 37 μm strainer to obtain the single cell suspension.

A single cell suspension of mouse mammary epithelial cells was isolated from mammary glands, as follows. A resected mammary gland was minced and placed in a tube containing standard culture medium, including 50 μg/ml gentamycin, collagenase, and hyaluronidase. The mammary gland was incubated for 2 hours at 37° C. in the tissue incubator. Following the incubation, the cell dissociation mixture was washed in advanced DMEM and centrifuged at 300 g for 5 minutes. Depending on the appearance of the pellet, in some cases it is desirable to resuspend the pellet in ammonium chloride to lyse contaminating red blood cells. Following, the pellet is resuspended in 0.25% Trypsin-EDTA, as above, for 5 minutes at 37° C. and then centrifuged at (300 g for 5 minutes). The supernatant was discarded and the pellet was resuspended in a 100 μg/ml DNase solution for 1 minute at room temperature. The resulting cells were passed through a 37 μm strainer to obtain the single cell suspension.

### Example 2: Seeding and Culturing Single Cell Suspensions of Mammary Epithelial Cells

Single cell suspensions obtained as described in Example 1 were reconstituted in 100% Matrigel™ (Corning) and seeded as domes in a well of a microplate, such that between about 1-2×10⁴ human cells or about 5×10³ mouse cells were seeded in each ˜25-40 μL dome. Each well received between 0.5-1 mL of any one of the various culture media described herein.

The plated single cell suspension of mammary epithelial cells formed into organoids that were passaged approximately every 7-days, with 2-3 full medium changes during such 7-day period. To passage the organoids, the Matrigel domes were broken apart by pipetting in pre-warmed 0.25% Trypsin-EDTA, incubating at 37° C. for ˜ 5-15 minutes and pipetting again. The trypsinization reaction was inactivated by washing in an equal volume of Hanks Balanced Salt Solution supplemented with 2% FBS. Depending on the density and size of organoids a 1:2-1:5 split was performed, with the cells seeded as domes, as described above. Alternatively, cells would be strained through a 37 μm strainer to obtain the single cell suspension and seeded in Matrigel domes as described above.

### Example 3: Staining Mammary Organoids

Organoids were recovered from Matrigel by incubating in Corning Cell Recovery Solution for 1 hour with rocking on ice. Organoids were then fixed in 4% paraformaldehyde (PFA) at room temperature for 1 hour and stored in PBS at 4° C. After PFA fixation, antigen retrieval was performed by boiling the organoids in sodium citrate buffer for 20 minutes at 96° C. Organoids were permeabilized in a 0.5% Triton-X-100 solution in PBS overnight at room temperature. Samples were blocked by incubating overnight in a solution of 5% goat serum.

Following fixation and permeabilization, the organoids were incubated sequentially in primary antibody and secondary antibody solutions (as summarized in the table below) overnight at room temperature. Samples were counterstained in a 2 μg/ml DAPI solution for 20 minutes and dehydrated through stepwise incubations in solutions with increasing methanol concentrations (50%, 80%, then 100% methanol). Organoids were embedded in a solution of 2:1 Benzyl Benzoate-Benzyl alcohol (BABB), transferred to Ibidi glass bottom chamber slides and imaged using a Leica SP8 confocal laser scanning microscope. Immunofluorescence (IF) buffer, consisting of 1% BSA, 0.1% cold fish skin gelatin, 0.2% Triton-X-100 and 0.05% Tween 20 in PBS, was used to wash organoids between each step.

| Type | Specificity | Supplier | Dilution |
|---|---|---|---|
| Primary | Guinea Pig IgG | Abcam (Cat | 1:500 |
| | anti-cytokeratin 8 | 194130) | |
| Primary | Mouse lgG3 anti- | Abcam (cat 7800) | 1:500 |
| | cytokeratin 14 | | |
| Secondary | Goat anti Guinea | Jackson | 1:500 |
| | Pig IgG (H + L) - | ImmunoResearch | |
| | AF488 | (cat 106-545-003) | |
| Secondary | Goat anti Mouse | Jackson | 1:500 |
| | IgG (H + L) - | ImmunoResearch | |
| | AF647 | (cat 115-605-003) | |

### Example 4: Forming Mouse Mammary Branched Organoids

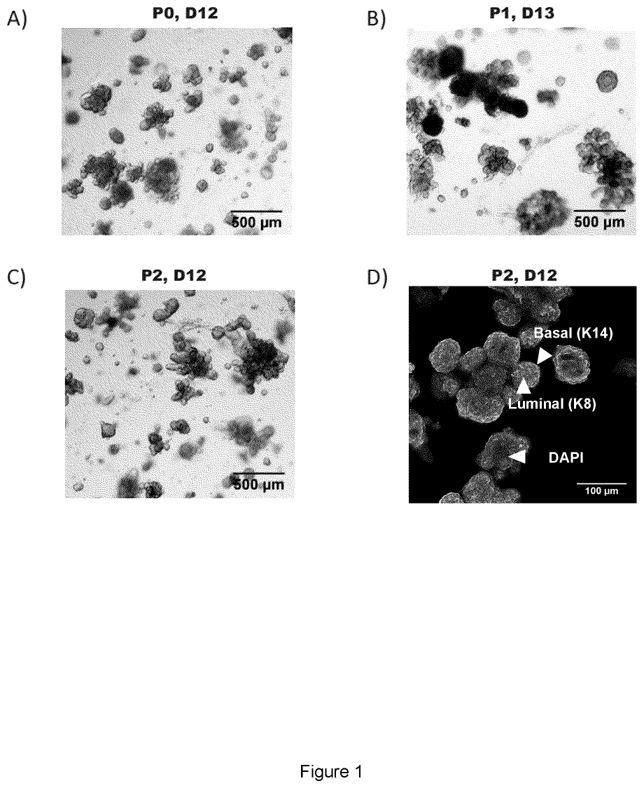

Single cell suspensions of mouse mammary epithelial cells were obtained in accordance with Example 1 and seeded and cultured essentially as described in Example 2 in a medium comprising a WNT signaling agonist (e.g. RSPO1), an inhibitor of BMP signaling (e.g. NOGGIN), and EGF (FIG. 1).

In three successive passages, the mouse mammary organoids formed in the foregoing medium consistently reproduced and maintained organoids characterized by high levels of branching, characteristic of organoids having substantial numbers of basal and other non-luminal cell types (FIGS. 1A, 1B, and 1C). After the second passage, the formed organoids were imaged using confocal microscopy as described in Example 3, and strong K14 expression was observed in highly branched organoids (FIG. 1D).

### Example 5: Forming Mouse Luminal-Biased Organoids in Organoid Media of this Disclosure

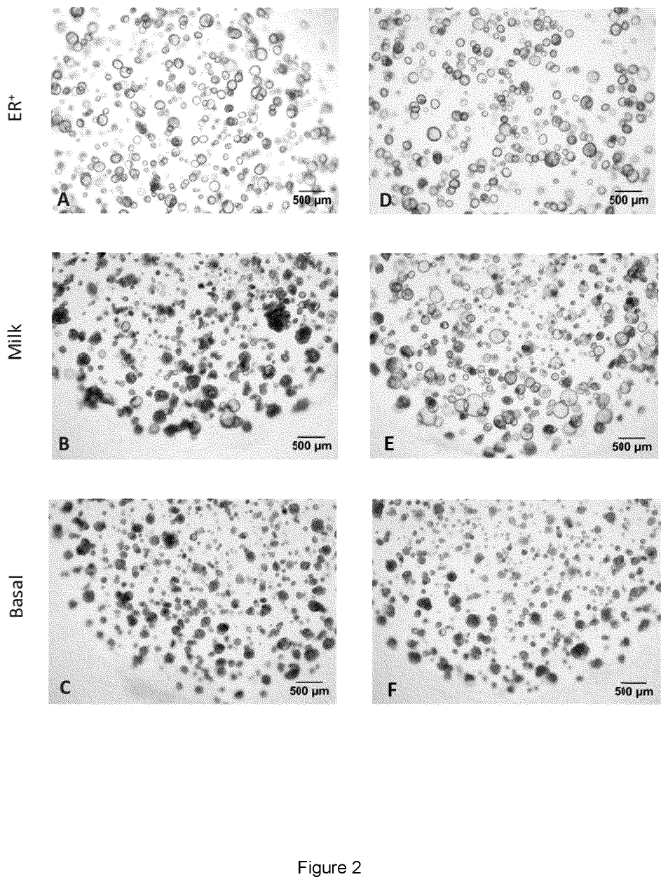

Single cell suspensions of mouse mammary epithelial cells were obtained in accordance with Example 1. The single cell suspension was sorted using a BD FACSAria™ Fusion flow cytometer into three populations of cells corresponding to the ER⁺, milk, and basal cell lineages, based on the differential expression of EpCAM, CD49f, and CD49b. Non-epithelial cells were further eliminated during the sorting strategy based on the expression of CD45, Ter119 and CD31. The sorted cells were seeded and cultured essentially as described in Example 2 in an organoid medium free of an exogenously-added inhibitor of BMP signaling and either comprising exogenously added RSPO-1 (˜100 ng/mL) or lacking exogenously added RSPO-1 (e.g. MammoCult™ Mouse Organoid Growth Medium, STEMCELL Technologies) (FIG. 2).

The ER⁺ lineage of mouse mammary epithelial cells formed into luminal organoids, having an apparent cystic organization, with generally the same efficiency in either of the tested medium formulations (FIGS. 2A & 2D). In contrast, the milk lineage of mouse mammary epithelial cells was biased toward a cystic organization in RSPO-1 deficient culture medium in comparison to a more mixed organization of basal, luminal and other cells when cultured in RSPO-1 containing culture medium (FIGS. 2B & 2E). As with the milk lineage, basal lineage cells appeared to bias toward a cystic organization in RSPO-1 deficient culture medium, although significant numbers of mixed organoids remained, in comparison to a predominately mixed organization when cultured in RSPO-1 containing culture medium (FIGS. 2C & 2F).

The observation that each of the three sorted lineages of mouse mammary epithelial cells could be biased toward a cystic organization (i.e. luminal organoids) based on medium formulation, prompted experiments to investigate the effect of different medium formulations on bulk (i.e. non-sorted) mammary epithelial cells.

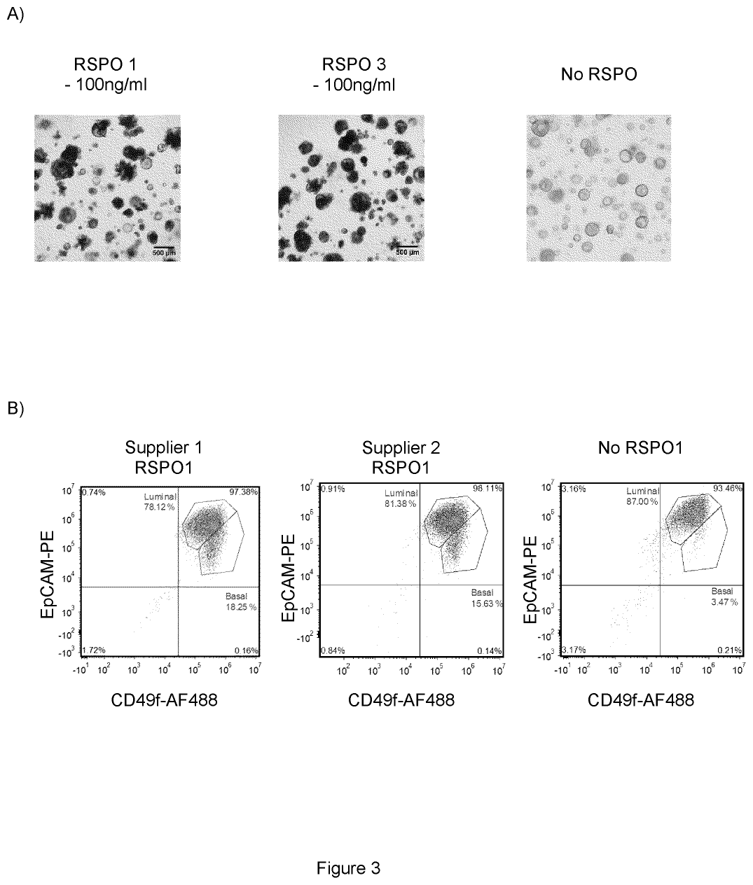

Organoid media were formulated to lack either exogenously added RSPO-1 or RSPO-3, and they were tested on single cell suspensions of bulk mammary epithelial cells, obtained in accordance with Example 1 and seeded and cultured as described in Example 2. Both RSPO-1 and RSPO-3 comprising media yielded a significant number of mixed organoids, but similar to FIG. 2 luminal-biased (or cystic) organoids were observed in organoid media free of an exogenously-added RSPO (or other Wnt agonist) (FIG. 3A)). Flow cytometry analysis of the cellular composition of organoids formed in either RSPO-comprising media or media lacking RSPO confirmed a reduction in the number of basal cells among organoids formed in the absence of WNT agonism (FIG. 3B).

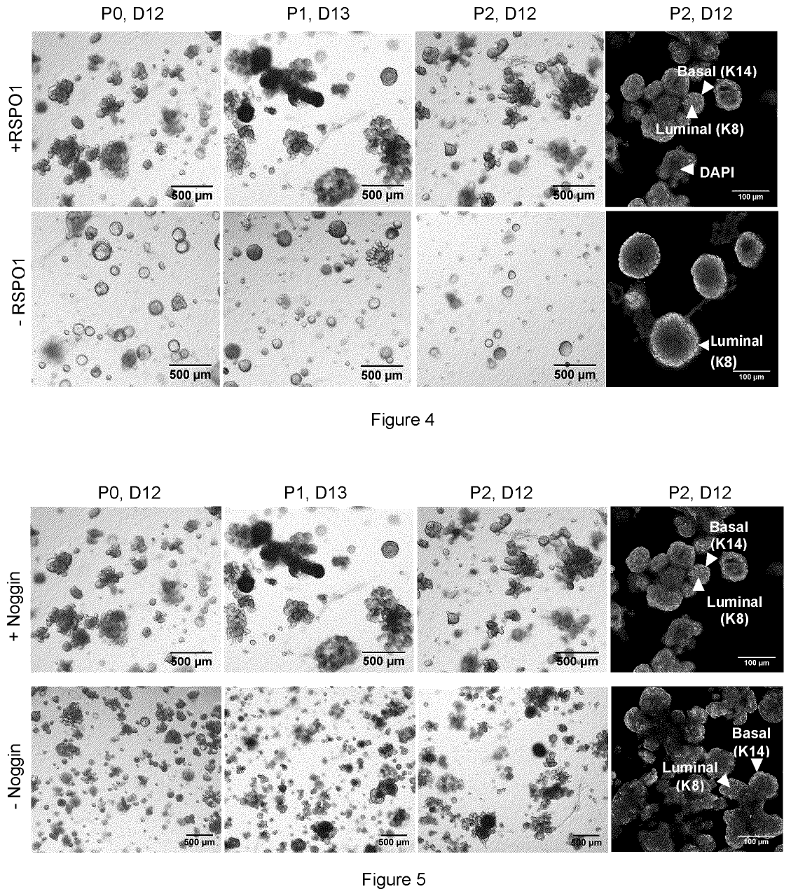

In a follow-up experiment, organoids were formed from single cell suspensions obtained in accordance with Example 1, and seeded and cultured as described in Example 2. The organoids were cultured for three successive passages in either the presence or absence of RSPO-1, and in each passage the organoids formed in the absence of RSPO-1 consistently reproduced and maintained a cystic organization devoid of branching, characteristic of luminal cell restricted organoids (FIG. 4; P0, P1 and P2). At the end of the second passage the formed organoids were processed and stained for K8, K14, and DAPI, as described in Example 3. Images obtained using confocal microscopy revealed a strong K8 expression and an apparent absence of K14 staining among the organoids formed in the absence of WNT agonism, which contrasts with the confocal microscopy images of organoids formed in the presence of WNT agonism (FIG. 4).

The foregoing experiments were performed in a medium that included an inhibitor of BMP signaling, thus the effect of BMP signaling inhibition on the formation of mouse mammary organoids was explored. Organoids were formed from single cell suspensions obtained in accordance with Example 1, and seeded and cultured as described in Example 2 in a medium comprising or lacking an exogenously-added inhibitor of BMP signaling (0 ng/mL or ˜100 ng/mL) (but included the WNT signaling agonist, RSPO-1). Brightfield images and confocal microscopy images taken across successive passages in the foregoing media formulations revealed significant numbers of branched organoids, comprising significant numbers of basal cells, regardless of the media formulation used (FIG. 5).

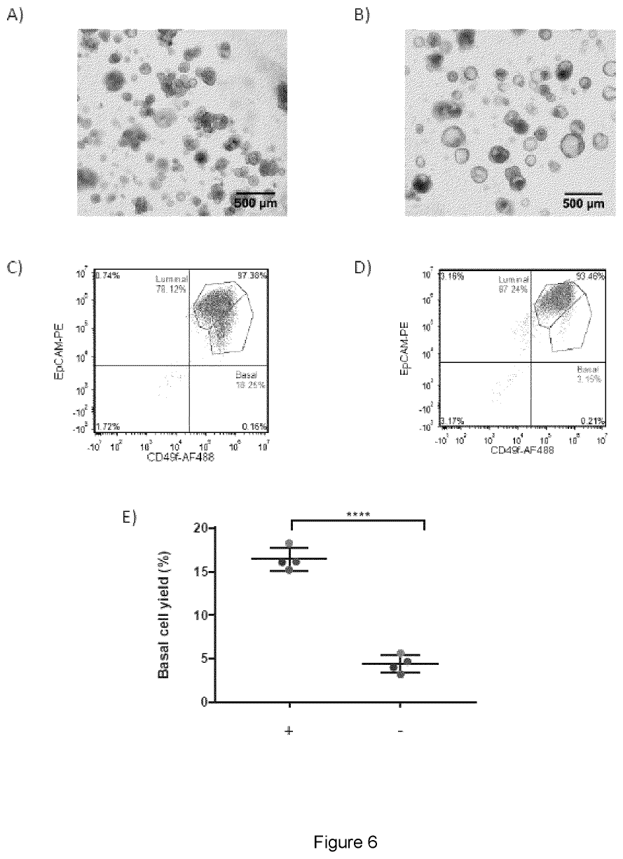

Next, the effect of the presence or absence of BMP signaling inhibition in the context of an organoid medium either including or lacking an exogenously-added WNT signaling agonist was explored. Organoids were formed from single cell suspensions obtained in accordance with Example 1, and seeded and cultured as described in Example 2. Based on 8 independent experiments using an organoid medium comprising ˜100 ng/mL NOGGIN and ˜100 ng/mL RSPO-1 (FIGS. 6A) and 6C)) or an organoid medium free of exogenously added NOGGIN- and RSPO-1 (FIGS. 6B) and 6D)) a significant reduction of basal cells with a concomitant increase in luminal cells was observed in the medium formulation free of the inhibitor of BMP signaling and of the WNT signaling agonist (FIG. 6E)

Based on the foregoing data, the absence of a WNT signaling agonist in an organoid medium greatly influences lineage balance (e.g. increases luminal cells) among mammary organoids, and the presence or absence of an inhibitor of BMP signaling appears to be dispensable for forming luminal-biased organoids.

### Example 6: Forming Human Mammary Branched Organoids

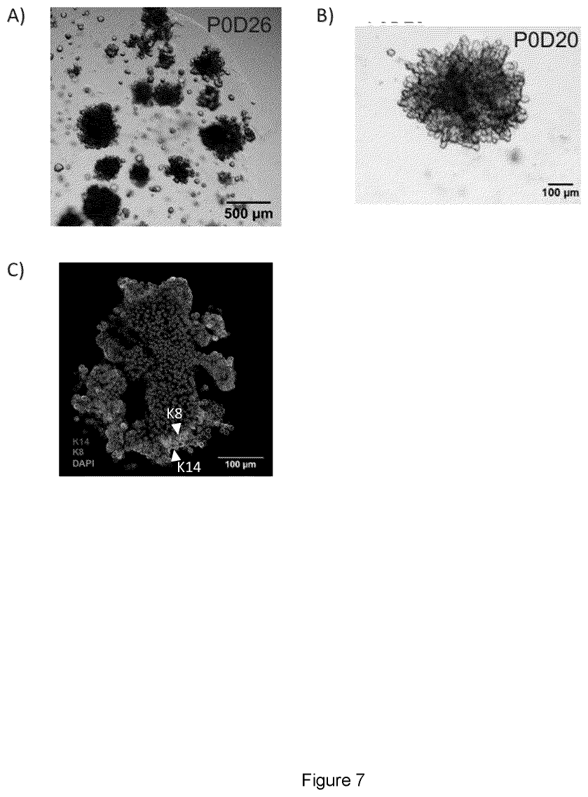

Single cell suspensions of human mammary epithelial cells were obtained in accordance with Example 1 and seeded and cultured essentially as described in Example 2 in a medium including RSPO-1, NOGGIN, and EGF (FIG. 7).

On day 26, the human mammary organoids formed in the foregoing medium consistently reproduced and maintained organoids characterized by high levels of branching, characteristic of organoids including high numbers of basal cells (FIGS. 7A, 7B, and 7C). The formed organoids were imaged using confocal microscopy, and strong K14 expression was observed in the highly branched organoids (FIG. 7C). The basal cells at the periphery of the lobes can be seen to stack one on the other. Instead of cohesive branching, the basal cells are marching away from the organoid in a single file and the result is “discohesive organoid branching”. The absence of a gland-forming unit is reminiscent of classic invasive lobular carcinoma.

### Example 7: Forming Human Luminal-Biased Organoids in Organoid Media of this Disclosure

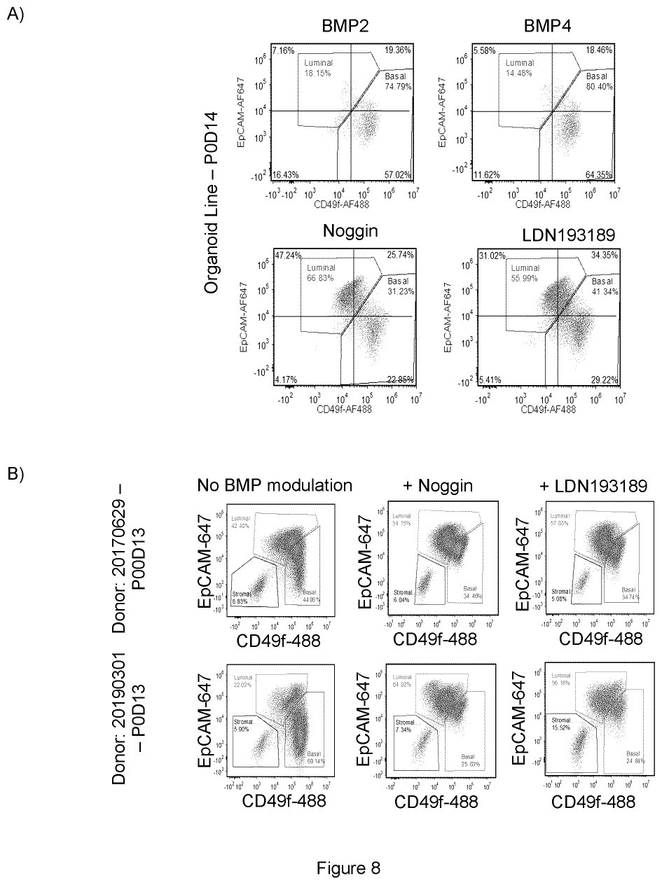

Single cell suspensions of human mammary epithelial cells were obtained in accordance with Example 1. The single cell suspension of cells was seeded and cultured essentially as described in Example 2 in an organoid medium (e.g. MammoCult™ Human Organoid Growth Medium, STEMCELL Technologies) testing the effects of different activators and inhibitors of BMP signaling (FIG. 8).

Organoids were formed in media comprising either BMP signaling activator, ˜50 ng/mL BMP2 or ˜50 ng/mL BMP4, and in media comprising either BMP signaling inhibitor, ˜25-200 ng/mL NOGGIN or ˜10-100 nM LDN193189. Just prior to the first passage, the formed organoids were dissociated and the cell lineage balance was analyzed by flow cytometry. Activation of signaling through the BMP pathway resulted in a significant increase in basal cells and reduction in luminal cells compared to organoids formed in media comprising an inhibitor of BMP signaling (FIG. 8A). Follow-up experiments in two different donor samples confirmed that inhibiting signaling through BMP decreased the number of basal cells while increasing the number of luminal cells in comparison to an organoid medium that included no BMP modulation (FIG. 8B).

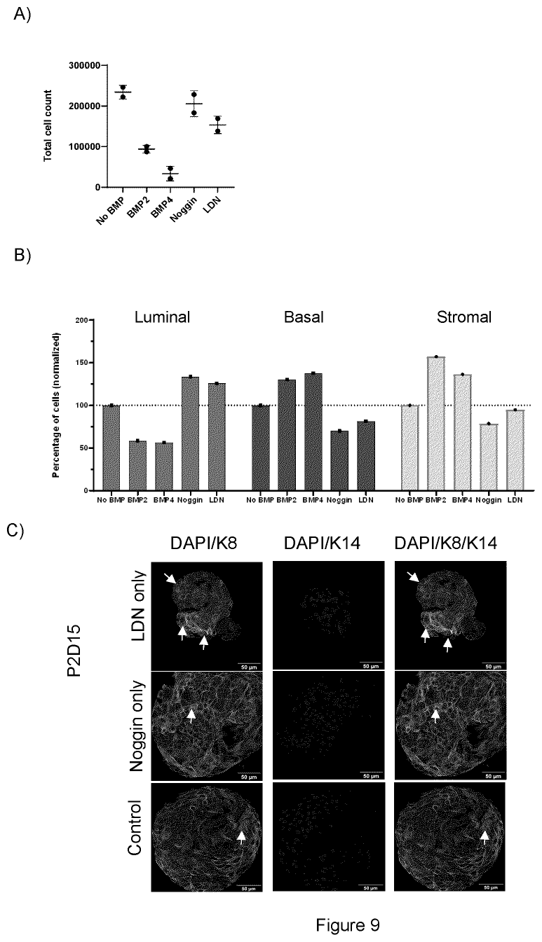

The effects of modulating signaling through BMP were analyzed across multiple experiments in terms of total cell count among formed organoids (FIG. 9A) and the lineage balance of the cells among formed organoids (FIG. 9B). Organoids formed in the foregoing conditions were analyzed for total cell count, and it was observed that activation of signaling through BMP, via either BMP2 or BMP4, resulted in a marked net loss of total cells in comparison to the condition where signaling through BMP was not modulated or was inhibited, via either NOGGIN or LDN193189 (FIG. 9A). Similar to the results observed in FIG. 8, the effects on cell lineage balance were confirmed for activation and inhibition of BMP signaling (FIG. 9B). The relative increase of K8⁺ luminal cells was confirmed by confocal microscopy for organoids formed in an organoid medium comprising an inhibitor of BMP signaling (FIG. 9C). Also, images taken by confocal microscopy revealed faint, if any, staining of K14⁺ cells in organoids formed in an organoid medium comprising an inhibitor of BMP signaling.

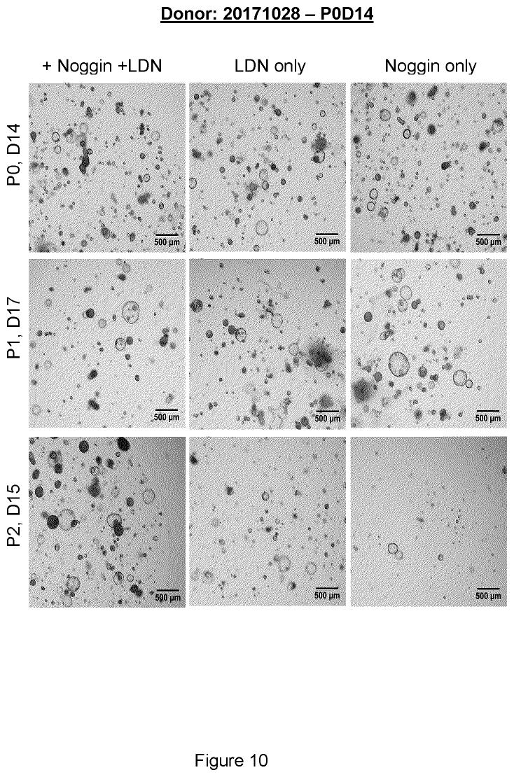

Last, potential additive effects of dual inhibition of signaling through BMP was explored (FIG. 10). Organoids were formed in media comprising both NOGGIN and LDN193189, or in media comprising either of NOGGIN or LDN193189. Examination of the cultures by brightfield microscopy revealed that organoid media comprising a single inhibitor of BMP signaling appeared sufficient to bias formation of luminal organoids from mammary epithelial cells (FIG. 10).

Overall, an absence of one or more inhibitors of BMP signaling in an organoid medium appears to yield organoids composed of significant numbers of luminal cells, and the addition of one or more inhibitors of BMP signaling in an organoid medium appears to increase the number of luminal cells while reducing the number of basal cells. In contrast, activating signaling through BMP appears to negatively influence the lineage balance of organoids if the formation of luminal-restricted or luminal-biased mammary organoids is desired.

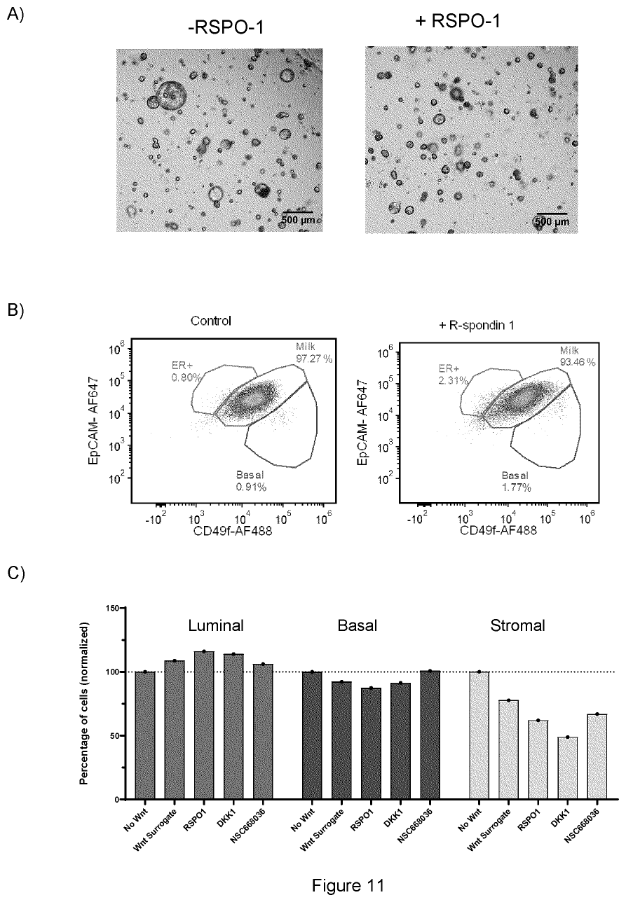

Given the important role of modulating BMP signaling on the lineage balance of human mammary organoids, potential effects of modulating WNT signaling on mammary organoid formation were next explored. Single cell suspensions of human mammary epithelial cells were obtained in accordance with Example 1. The single cell suspension of cells was seeded and cultured essentially as described in Example 2 in an organoid medium testing the effects of different activators (˜0.1-1 nM WNT surrogate or ˜20-200 ng/mL RSPO-1) and inhibitors (˜50 ng/mL DKK1 or ˜200 nM NSC) of WNT signaling (FIG. 11).

A luminal-promoting organoid medium was formulated (e.g. MammoCult™ Human Organoid Growth Medium, STEMCELL Technologies), and was either supplemented with exogenously-added RSPO-1 or lacked exogenously-added RSPO-1. Brightfield images of the formed organoids did not reveal a significant increase or reduction of luminal-biased organoids between the two formulations tested (FIG. 11A). This observation was confirmed after analyzing the cells of organoids formed in either condition by flow cytometry (FIG. 11B). Compiling flow cytometry data of multiple organoid forming experiments similarly suggested that modulating WNT signaling had little effect on lineage balance of the cells of the analyzed organoids (FIG. 11C). There appeared to be more or less equivalent percentages of luminal and basal cells regardless of whether the organoid medium included WNT signaling agonists, WNT signaling antagonists, or no modulation of WNT signaling. Thus, WNT signaling agonism and/or antagonism appears to be dispensable for the formation of luminal-restricted or luminal-biased human mammary organoids from mammary epithelial cells.

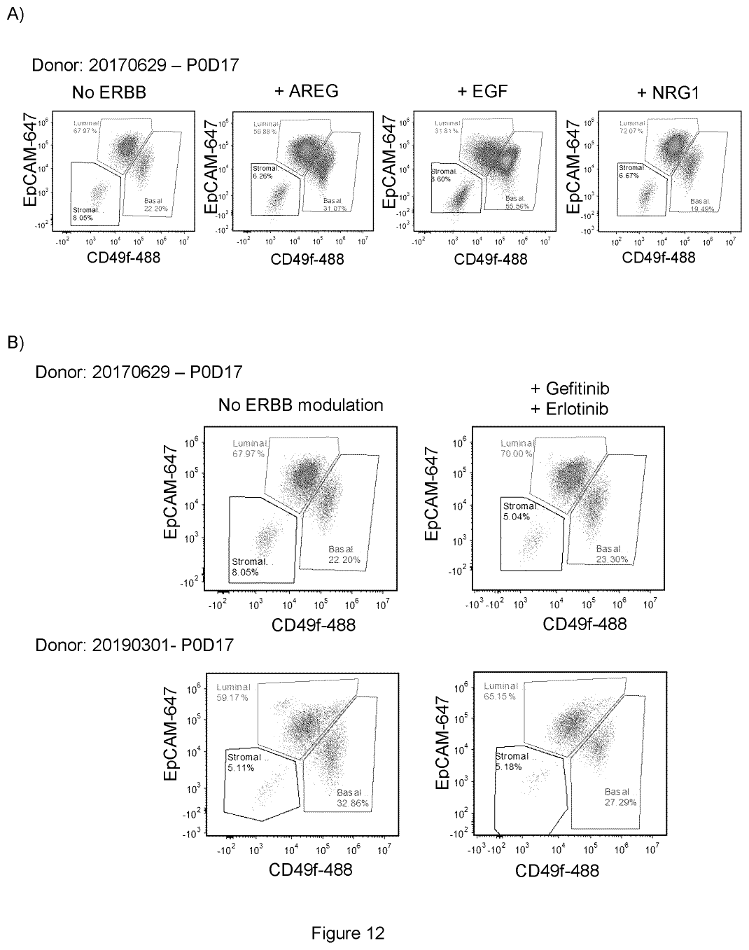

Given that WNT signaling modulation appeared dispensable for forming luminal-restricted or luminal-biased human mammary organoids, and that inhibition of BMP signaling appeared to tip cell lineage balance in favour of luminal cells, the effect of modulating signaling through ERBB family members on human mammary organoid formation was next explored. Single cell suspensions of human mammary epithelial cells were obtained in accordance with Example 1. The single cell suspension of cells was seeded and cultured essentially as described in Example 2 in an organoid medium (e.g. MammoCult™ Human Organoid Growth Medium, STEMCELL Technologies) testing ligands of different ERBB receptor family members (FIG. 12).

Organoids were formed in media comprising either no ligand of an ERBB receptor family member, or comprising ˜10-50 ng/mL amphiregulin (AREG), ˜10-50 ng/mL epidermal growth factor (EGF), or ˜50-100 ng/mL heregulin/neuregulin 1 (NRG1). Just prior to the first passage, the formed organoids were dissociated and the cell lineage balance was analyzed by flow cytometry. Whereas AREG and NRG1 appeared to have a minor or no effect on cell lineage balance, EGF appeared to significantly reduce the number of luminal cells while significantly increasing the number of basal cells among the formed organoids (FIG. 12A). Follow-up experiments in two donor samples tested the effects of inhibiting signaling through the EGF receptor via treatment with ˜100 nM Gefitinib and ˜100 nM Erlotinib, and it appeared that this treatment had negligible or no effect on cell lineage balance (FIG. 12B).

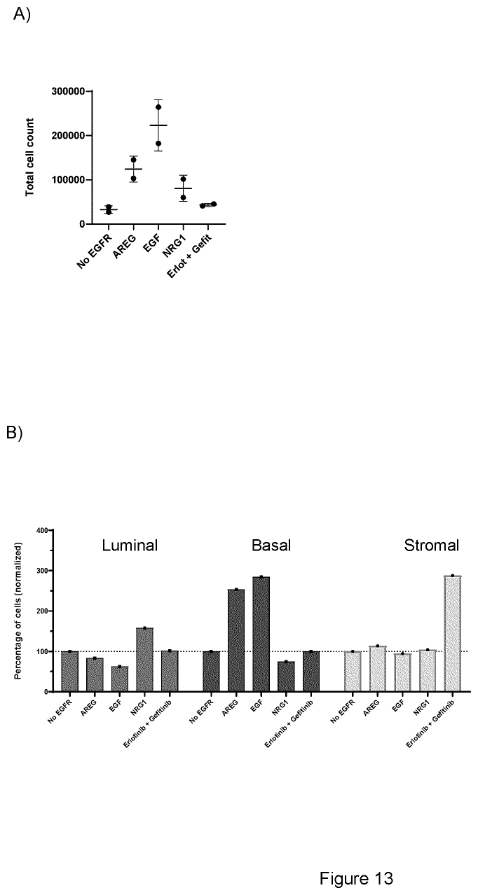

The effects of modulating signaling through an ERBB receptor family member were analyzed across multiple experiments in terms of total cell count among formed organoids (FIG. 13A) and the lineage balance of the cells among formed organoids (FIG. 13B). Organoids formed in the foregoing conditions were analyzed for total cell count, and it was observed that inclusion of an activator of signaling through an ERBB family member resulted in a net increase in the total number of cells among formed mammary organoids in comparison to a control condition that included no modulation of ERBB signaling (FIG. 13A). The highest mitogenic activity was observed where either EGF or AREG were included in an organoid medium. Similar to the results observed in FIG. 12, the effects on cell lineage balance were confirmed for the various ERBB receptor family ligands tested (FIG. 13B). Whereas EGF resulted in the greatest reduction in luminal cells and the greatest increase in basal cells, AREG appeared to have minimal impact on luminal cell percentages but profound impact on increasing basal cell percentages. In contrast, inclusion of NRG1 in an organoid medium appeared to yield a higher percentage of luminal cells and a reduction in the percentage of basal cells.

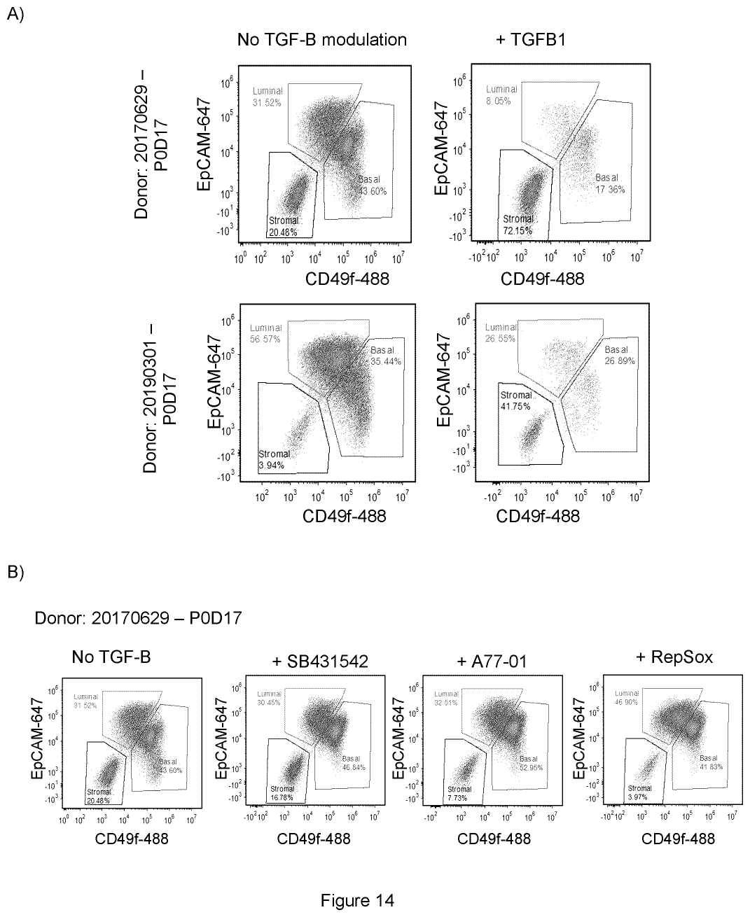

In a last set of experiments, the effects of modulating signaling through TGFβ on human mammary organoid formation was explored. Single cell suspensions of human mammary epithelial cells were obtained in accordance with Example 1. The single cell suspension of cells was seeded and cultured essentially as described in Example 2 in an organoid medium (e.g. MammoCult™ Human Organoid Growth Medium, STEMCELL Technologies) testing the effects of a ligand of a TGFβ receptor or of inhibitors of signaling through TGFβ (FIG. 14).

Organoids were formed in media comprising either no ligand of a TGFβ receptor, or comprising ˜50 ng/mL TGFβ1 (or TGFβ3, data not shown but results consistent with TGF β1). Just prior to the first passage, the formed organoids were dissociated and the cell lineage balance was analyzed by flow cytometry. The presence of TGFβ1 in an organoid medium appeared to have an overall negative impact on the percentage of luminal and basal cells, while greatly increasing the proportion of stromal cells among the formed mammary organoids (FIG. 14A).

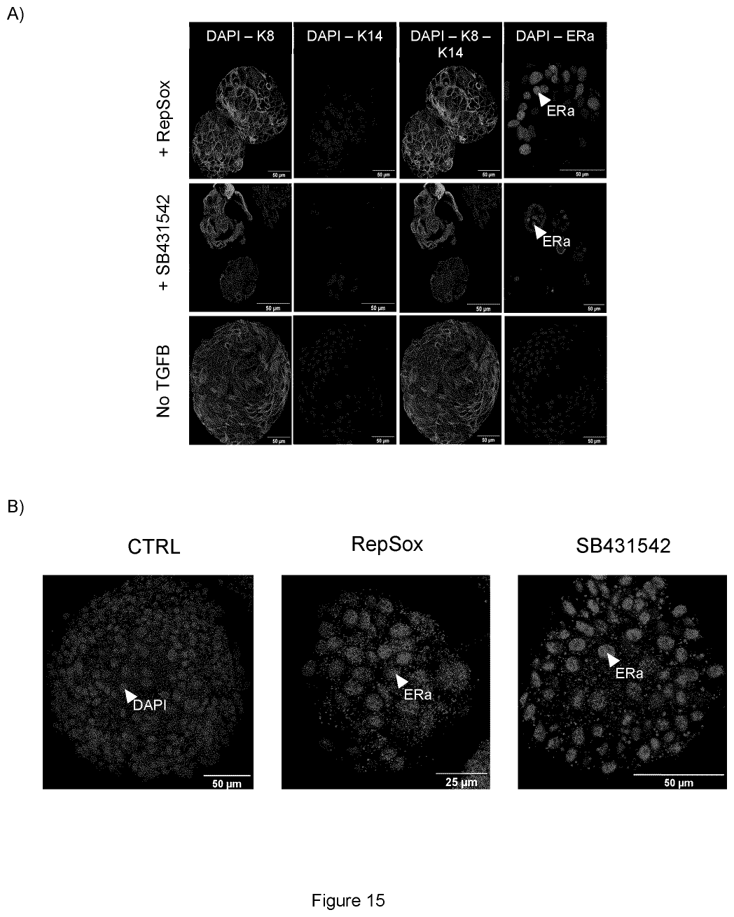

Given that inclusion of TGFβ1 in an organoid medium negatively impacted the formation of luminal-restricted or luminal-biased human mammary organoids, potential roles of inhibiting signaling through a TGFβ receptor were explored. Inclusion of either ˜10 μM SB431542, ˜200-500 nM A77-01, or ˜5-25 RepSox UM) in an organoid medium appeared to reduce the proportion of stromal cells while either maintaining or improving the proportions of luminal cells (FIG. 14B). Based on these results including an inhibitor of TGFβ signaling, such as RepSox, in an organoid medium may increase the proportion of luminal cells in comparison to an organoid medium that does not include a modulator of signaling through a TGFβ receptor. However, a potentially more interesting finding suggested that a transient exposure to an inhibitor of TGFβ signaling (after organoids have formed as described above) promoted the localization of estrogen receptor within nuclei, rather than within the cytoplasm (FIG. 15). These results were confirmed for both SB43154 and RepSox in both human (FIG. 15A) and mouse (FIG. 15B) mammary organoids.

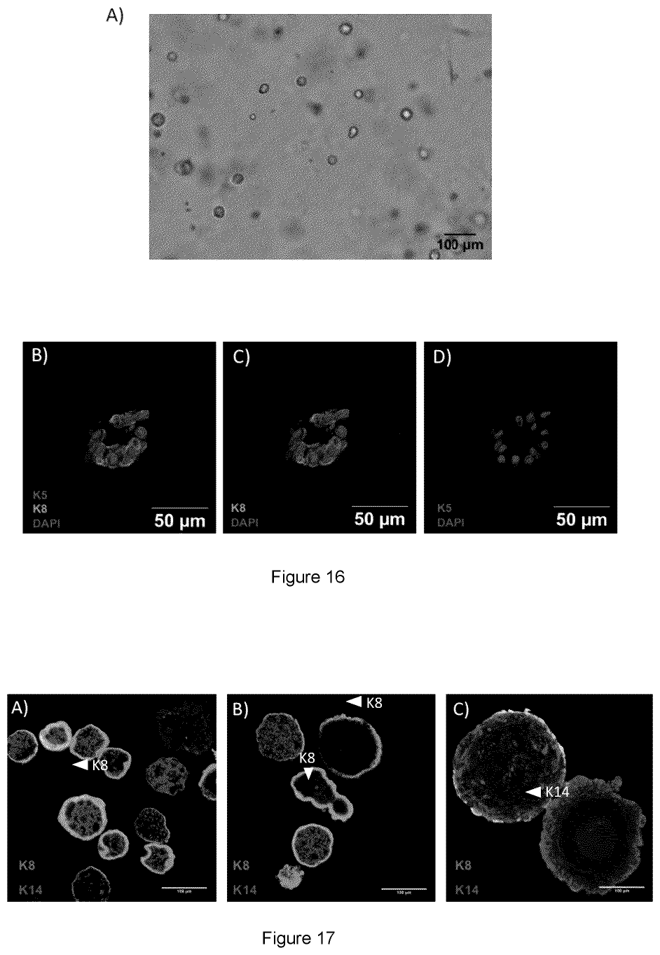

In a follow-up experiment, an organoid medium was formulated to lack both exogenously added RSPO-1 and Noggin. This medium was tested on single cell suspensions, that were obtained in accordance with Example 1, and that were seeded and cultured as described in Example 2. On day 10 after the first passage, the organoids formed without exposure to exogenously added RSPO-1 and Noggin exhibited the cystic morphology and absence of branching that is characteristic of luminal organoids (FIG. 16A). Confocal microscopy on the mammary organoids formed under these culture conditions verified the presence of luminal cell marker expression (K8) and an apparent lack of basal cell marker expression (K5) (FIGS. 16B, 16C, and 16D).

### Example 8: Additional Factors that Promote Formation of Mammary Luminal Organoids

Other candidate factors were tested in organoid media to determine their effects on biasing the formation of mammary organoids restricted to specific lineages of mammary epithelial cells. Single cell suspensions of human mammary epithelial cells were obtained in accordance with Example 1. The single cell suspension of cells was seeded and cultured essentially as described in Example 2 in an organoid medium (e.g. MammoCult™ Human Organoid Growth Medium, STEMCELL Technologies) supplemented with either 50 ng/mL Neuregulin (NRG3), 100 ng/mL anti mullerian hormone (AMH), or 50 ng/mL of Granulocyte-macrophage Colony Stimulating Factor (GM-CSF).

Day 17 organoids were stained for either K14 or K8 and imaged by confocal microscopy (FIG. 17). Inclusion of either NRG3 or AMH in the organoid medium appeared to bias the culture of mammary epithelial cells to form luminal-restricted organoids (FIG. 17A and FIG. 17B), while inclusion of GM-CSF in the organoid medium appeared to bias the culture of mammary epithelial cells to form basal-restricted organoids (FIG. 17C).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of forming mammary organoids from isolated mammary epithelial cells, comprising:
a) contacting the mammary epithelial cells with an organoid medium free of one or both of an exogenously-added WNT signaling agonist and/or an inhibitor of BMP signaling; and
b) culturing the mammary epithelial cells in the organoid medium for a time sufficient to form a first population of organoids enriched for organoids composed of more luminal cells than non-luminal cells,
wherein the organoid medium includes a basal medium and one or both of a ligand of ERBB1 and/or a ligand of ERBB4.

2. The method of claim 1, wherein the organoid medium includes the inhibitor of BMP signaling but not the WNT signaling agonist, or the organoid medium includes the WNT signaling agonist but not the inhibitor of BMP signaling.

3-6. (canceled)

7. The method of claim 1, wherein the ligand of ERBB1 is not EGF or TGFalpha.

8. The method of claim 7, wherein the ligand of ERBB1 is amphiregulin, and the ligand of ERBB4 is neuregulin 1 and/or neuregulin 3.

9. The method of claim 1, wherein the ligand of ERBB4 is also a ligand of a different ERBB receptor family member.

10. (canceled)

11. The method of claim 1, wherein the organoid medium is free of an exogenously added sex hormone.

12. (canceled)

13. The method of claim 1, wherein non-luminal cells are one or more of basal cells, stromal cells, hematopoietic cells, and endothelial cells.

14. The method of claim 1, wherein the organoids are composed of 50% or more luminal cells.

15. The method of claim 1, further comprising culturing the first population of organoids in a modified organoid medium to subvert the first population of organoids into a second population of organoids, wherein the modified organoid medium is supplemented with EGF and/or the modified organoid medium is supplemented with one or both of a WNT signaling agonist and an inhibitor of BMP signaling.

16-17. (canceled)

18. The method of claim 15, wherein the second population of organoids are composed of more basal cells and fewer luminal cells then if one or both of the WNT signaling agonist and/or the inhibitor of BMP signaling are not added to the organoid medium.

19-20. (canceled)

21. The method of claim 1, further comprising contacting the first population of organoids with an inhibitor of TGF-beta.

22. The method of claim 21, further comprising obtaining nuclear localization of ER.

23. A mammary organoid medium, comprising: a basal medium and one or both of a ligand of ERBB1 and a ligand of ERBB4; and lacking one or both of an exogenously-added WNT signaling agonist and an inhibitor of BMP signaling.

24. The medium of claim 23, wherein the organoid medium includes the inhibitor of BMP signaling but not the WNT signaling agonist, or the organoid medium includes the WNT signaling agonist but not the inhibitor of BMP signaling.

25. (canceled)

26. The medium of claim 23, wherein the WNT signaling agonist is an R-spondin, a WNT protein, or an engineered mimetic of either of the foregoing and the inhibitor of BMP signaling is one or more of Noggin, chordin, follistatin, LDN193189, or dorsomorphin.

27. (canceled)

28. (canceled)

29. The medium of claim 23, wherein the ligand of ERBB1 is not EGF or TGFalpha.

30. The medium of claim 23, wherein the ligand of ERBB1 is amphiregulin and the ligand of ERBB4 is neuregulin 1 or neuregulin 3.

31. The medium of claim 23, wherein the ligand of ERBB4 is also a ligand of a different ERBB receptor family member.

32. (canceled)

33. The medium of claim 23, wherein the organoid medium is free of an exogenously added sex hormone.

34-38. (canceled)
